(19) United States
(12) Patent Application Publication
(10) Pub. No.: US 2006/0058369 A1
(43) Pub. Date: Mar. 16, 2006

(54) Therapeutic methods employing PAI-1 inhibitors and transgenic non-human animal for screening candidate PAI-1 inhibitors

(75) Inventors: Douglas E. Vaughan, Nashville, TN (US); Mesut Eren, Nashville, TN (US)

Correspondence Address: JENKINS, WILSON & TAYLOR, P. A., 3100 TOWER BLVD, SUITE 1400, DURHAM, NC, 27707, US

(73) Assignee: Vanderbilt University

(21) Appl. No.: 11/171,083

(22) Filed: Jun. 29, 2005

Related U.S. Application Data
(63) Continuation-in-part of application No. 10/368,995, filed on Feb. 19, 2003.
(60) Provisional application No. 60/358,061, filed on Feb. 19, 2002.

(51) Int. Cl. A61K 31/404

(52) U.S. Cl. 514/416

(57) ABSTRACT

A method of treating a warm-blooded vertebrate animal having a medical condition in need of treatment with a composition that exhibits PAI-1 inhibition activity is disclosed. The method includes the steps of administering a treatment effective amount of the composition to a warm-blooded vertebrate animal having a medical condition selected from the group consisting of alopecia, undesired weight loss, Alzheimer's Disease, systemic amyloidosis, myelofibrosis, male pattern baldness, glomerulosclerosis, veno-occlusive disease (VOD), pulmonary fibrosis, obesity, non-alcoholic steatohepatitis (NASH), osteoporosis, osteopenia, polycystic ovarian syndrome (PCOS), and combinations thereof; and observing an improvement in the medical condition in the warm-blooded vertebrate animal having the medical condition. A method of testing a candidate composition for PAI-1 inhibition activity is also disclosed. The method includes the steps of obtaining a transgenic non-human warm blooded vertebrate animal having incorporated into its genome a PAI-1 gene encoding a biologically active PAI-1 polypeptide, the PAI-1 gene being present in the genome in a copy number effective to confer overexpression in the transgenic non-human animal of the PAI-1 polypeptide; administering the composition to the transgenic non-human animal; and observing the transgenic non-human animal for determination of a change in the transgenic non-human animal indicative of inhibition of the activity of PAI-1. A transgenic non-human animal useful is such a method is also disclosed, as is a PAI-1 transgene construct encoding a biologically active PAI-1 polypeptide useful for preparing the transgenic non-human animal.

### CROSS REFERENCE TO RELATED APPLICATIONS

This application is a continuation-in-part of U.S. patent application Ser. No. 10/368,995, filed Feb. 19, 2003, which itself is based on and claims priority to U.S. Provisional Application Ser. No. 60/358,061, filed Feb. 19, 2002, the disclosures of both of which are herein incorporated by reference in their entireties.

### TECHNICAL FIELD

The presently disclosed subject matter relates, in general, to therapeutic methods for warm-blooded vertebrate animals and to transgenes and non-human transgenic animals. More particularly, the presently disclosed subject matter relates to a construct comprising a plasminogen activator inhibitor-1 (abbreviated as PAI-1) gene encoding a biologically active PAI-1 polypeptide and a vector. Also, the presently disclosed subject matter relates to a transgenic non-human vertebrate animal having such a PAI-1 gene incorporated into its genome, for instance, a transgenic mouse, and a method of employing such transgenic animals to test candidate compositions to determine if they have PAI-1 inhibition activity. Furthermore, the presently disclosed subject matter relates to employing PAI-1 activity-inhibiting compositions in a method of treating warm-blooded vertebrate animals.

TABLE OF ABBREVIATIONS

| | |
|---|---|
| ACEI | angiotensin converting enzyme inhibitor |
| AIIRA | angiotensin II receptor antagonist |
| BAC | bacterial artificial chromosome |
| COPD | chronic obstructive pulmonary disease |
| ECM | extracellular matrix |
| kDa | kiltodalton(s) |
| μM | micromolar |
| mCT | micro-computed tomography |
| MMP | matrix metallo-protease |
| mPPET-1 | mouse preproendothelin-1 |
| NASH | non-alcoholic steatohepatitis |
| p5.9 | plasmid containing the mouse preproendothelin-1 |
| | (mPPET-1) gene promoter |
| PA | plasminogen activator |
| PAI | plasminogen activator inhibitor |
| PAI-1 | plasminogen activator inhibitor-1 |
| PAI-1.stab | a stable, constitutively active form of human |
| | plasminogen activator inhibitor-1, or a |
| | transgenic mouse line overexpressing the |
| | same |
| PCOS | polycystic ovarian syndrome |
| R346A-stab | a human PAI-1 with a reactive site mutation, or a |
| | transgenic mouse line overexpressing the |
| | same |
| RCL | reactive center loop |
| tPA | tissue-type PA |
| uPA | urokinase-type PA |
| VN | vitronectin |
| VNBS | VN binding site |
| VOD | veno-occlusive disease |
| YAC | yeast artificial chromosome |

### BACKGROUND ART

The plasminogen activator (PA) system has an important role in controlling endogenous fibrosis and regulating the extracellular matrix (ECM) proteolysis relevant to tissue remodeling (Gabazza et al., 1999). The tissue-type PA (tPA) and urokinase-type PA (uPA) convert plasminogen to plasmin, which enhances proteolytic degradation of the ECM. An important mechanism in the regulation of PA activity is the inhibition of uPA or tPA by three major inhibitors, which are PAI-1, PAI-2, and PAI-3 (Kruithof, 1988). Thus, as is well known, the plasminogen activator/plasmin system plays a critical role in fibrinolysis, cellular migration, and matrix remodeling. More specifically, Stefansson and Lawrence, 1996 describes how PAI-1 blocks cell migration. Furthermore, Nar et al., 2000 describes the structure of PAI-1. Carmeliet et al., 1993 describes mice lacking sufficient PAI-1.

To elaborate, plasminogen is converted to its active form, plasmin, by the serine proteases tissue-type plasminogen activator (t-PA) and urokinase-type plasminogen activator (u-PA; Sprengers and Kluft, 1987). Plasmin has a broad spectrum of proteolytic activities such as degradation of fibrin and activation of matrix metallo-proteases (MMPs), which degrade extracellular matrix (ECM) and play important roles in tissue remodeling. The t-PA activated plasminogen system is primarily responsible for degradation of fibrin. The balance between plasminogen activators (PA) and plasminogen activator inhibitor-1 (PAI-1) predominantly determines the plasma fibrinolytic activity (Rosenberg and Aird, 1999). The u-PA activated plasminogen system functions in cell migration and tissue remodeling. The activation of the plasminogen system is regulated either by inhibition of t-PA or u-PA by plasminogen activator inhibitor type-1 (PAI-1; Francis et al., 1988) or by inhibition of plasmin by α₂-antiplasmin (Booth, 1994 at pages 699-717).

Plasma PAI-1 appears to mainly originate from the vascular endothelium, adipose tissue, and the liver (Loskutoff et al., 1986; Samad et al., 1996; Chomiki et al., 1994) and large quantities of which is stored by platelets and secreted upon platelet aggregation (Declerck et al., 1988b). PAI-1 and t-PA exist in plasma in 4:1 molar ratio (Vaughan et al., 1997) and PAI-1 in circulation has a T_{1/2} of approximately 5 minutes and is removed via a hepatic clearance mechanism (Vaughan et al., 1990).

Only a fraction of the secreted, active PAI-1 reacts with plasma t-PA, and forms inert, covalent complexes. The majority of PAI-1 in plasma and PAI-1 in the extracellular matrix of blood vessels binds to the 75 kilodalton (kDa) glycoprotein vitronectin (VN). The PAI-1-vitronectin complex might represent the physiologically relevant form of the inhibitor in the extracellular matrix (Keijer et al., 1991).

PAI-1 production is stimulated by a number of factors such as inflammatory cytokines, e.g. interleukin-I (IL-1; Emeis and Kooistra, 1986) and tumor necrosis factor α (TNFα), transforming growth factor β (TGFβ; Sawdey et al., 1989), epidermal growth factor (EGF), thrombin (Dichek and Quertermous, 1989), and insulin (Alessi et al., 1988). The infusion of endotoxin has also stimulated PAI-1 levels in plasma (Emeis and Kooistra, 1986; Colucci et al., 1985). Angiotensin II (Ang II) and angiotensin IV (Ang IV) also stimulate induction of PAI-1 transcription in vascular tissue in vitro and in vivo (Vaughan et al., 1995; Feener et al., 1995).

The reactive center loop (RCL) of PAI-1 serves as the suicide inhibitory substrate for t-PA and u-PA by forming a covalent complex with PAs after its RCL is cleaved at ³⁴⁶Arg-³⁴⁷Met bond (P1-P1′; Aertgeerts et al., 1994; Kruithof et al., 1984). PAI-1 spontaneously acquires a thermodynamically more stable but functionally inactive latent form (Declerck et al., 1988a). A series of amino acid substitutions (N150H, K154T, Q301P, Q315L, and M354I) resulted in stabilization of reactive center loop of human PAI-1 in the active conformation (referred to as PAI-1.stab) and extended the T_{1/2} of the enzyme from 2.5 hrs to >145 hrs at 37° C. in vitro (Berkenpas et al., 1995). Clinical evidence linking PAI-1 with arterial and venous thrombosis stresses the physiological importance of PAI-1 (Wiman et al., 265-270, 1985; Auwerx et al., 1988; Margaglione et al., 1994; Thogersen et al., 1988; Juhan-Vague et al., 1987).

Despite the above-described efforts, there remains a need in the art for further characterization of the biological role of PAI-1. An animal model to facilitate such characterization is also needed. The presently disclosed subject matter addresses these and other needs in the art.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

A method of treating a warm-blooded vertebrate animal having a medical condition in need of treatment with a composition that exhibits PAI-1 inhibition activity is disclosed. The method comprises administering a treatment effective amount of the composition to a warm-blooded animal having a medical condition selected from the group consisting of alopecia, undesired weight loss, Alzheimer's disease, systemic amyloidosis, myelofibrosis, nephrosclerosis, pattern baldness, veno-occlusive disease (VOD), obesity, non-alcoholic steatohepatitis (NASH), osteoporosis, osteopenia, polycystic ovarian syndrome (PCOS), and combinations thereof; and observing an improvement in the medical condition in the warm-blooded animal having the medical condition.

Also provided is a method of hepatoprotection. In some embodiments, the method comprises: (a) administering a treatment effective amount of a composition that exhibits PAI-1 inhibition activity to a warm-blooded vertebrate animal in need of hepatoprotection and having a medical condition associated with PAI-1 biological activity; and (b) observing an improvement in the medical condition indicative of inhibition activity of PAI-1 in the warm-blooded animal having the medical condition.

A transgenic non-human warm-blooded vertebrate animal having incorporated into its genome a PAI-1 gene encoding a biologically active PAI-1 polypeptide is also disclosed. In a preferred embodiment, the PAI-1 gene is present in the genome of the animal in a copy number effective to confer overexpression in the animal of the PAI-1 polypeptide.

A transgene construct comprising an isolated PAI-1 gene encoding a biologically active PAI-1 polypeptide cloned into a vector is also disclosed.

A method of testing a candidate composition for PAI-1 inhibition activity is also disclosed. The method comprises obtaining a transgenic non-human warm blooded vertebrate animal having incorporated into its genome a PAI-1 gene encoding a biologically active PAI-1 polypeptide, the PAI-1 gene being present in the animal's genome in a copy number effective to confer overexpression in the animal of the PAI-1 polypeptide; administering the composition to the animal; and observing the animal for determination of a change in the animal indicative of inhibition of the activity of PAI-1.

The presently disclosed subject matter also provides a method of treating a warm-blooded vertebrate animal having veno-occlusive disease (VOD) associated with PAI-1 biological activity. In some embodiments, the method comprises (a) administering a treatment effective amount of a composition that exhibits PAI-1 inhibition activity to a warm-blooded animal having veno-occlusive disease (VOD) associated with PAI-1 biological activity; and (b) observing an improvement in the VOD indicative of inhibition activity of PAI-1 in the warm-blooded animal. In some embodiments, the warm-blooded vertebrate animal is a human. In some embodiments, the composition that exhibits PAI-1 inhibition activity comprises tiplaxtinin (PAI-039; 2-{1-Benzyl-5-[4-(trifluoromethoxy)phenyl]-1H-indol-3-yl}-2-oxoacetic acid) or a pharmaceutically acceptable salt thereof.

Accordingly, it is an object of the presently disclosed subject matter to provide a novel method of treating disorders with a PAI-1 activity-inhibiting composition. This and other objects are achieved in whole or in part by the presently disclosed subject matter.

An object of the presently disclosed subject matter having been stated above, other objects will become evident as the description proceeds when taken in connection with the accompanying Figures and Examples as best described below.

### BRIEF DESCRIPTION OF THE FIGURES

FIG. 1 depicts a representative embodiment of a stable PAI-1 transgene construct.

FIGS. 2A-2C is a photograph comparing transgenic mice PAI-1.stab +/+ (FIG. 2A) and PAI-1.stab +/− (FIG. 2C), with a wild type mouse (FIG. 2B) to illustrate the extent of epidermal phenotype in the transgenic mice.

FIGS. 3A-3D is a set of photographs comparing hemotoxylin and eosin stained spleen sections from a wild type mouse (FIGS. 3A and 3C) and a transgenic mouse (FIGS. 3B and 3D). FIGS. 3A and 3B are at 10× magnification and FIGS. 3C and 3D are at 60× magnification.

FIGS. 4A and 4B depict Masson's trichrome-stained liver tissue isolated from wild type (FIG. 4A) or PAI-1 deficient (FIG. 4B) mice.

FIGS. 5 and 6 depict the effects of chronic nitric oxide synthase inhibition with an oral inhibitor (L-NAME) on plasma markers of hepatic injury in wild type and PAI-1 deficient mice.

FIG. 5 is a bar graph of data depicting the effect of L-NAME on total bilirubin levels, expressed on the y-axis in mg/dL.

FIG. 6 is a bar graph depicting the effect of L-NAME on aspartate amino transferase (AST) levels, expressed on the y-axis in U/mL.

FIGS. 7A-9B depict visualizations of the morphological and microarchitectural features of the skeletal systems of wild type and PAI-1 overexpressing transgenic mice.

FIGS. 7A-7C are X-ray images of wild type (FIG. 7A), stable PAI-1 (PAI-1.stab; FIG. 7B), and stable PAI-1-reactive site mutant (R346A-stab; FIG. 7C) transgenic mice.

FIGS. 8A and 8B are iso-surface images of wild type (FIG. 8A) and stable PAI-1 transgenic (FIG. 8B) mice. This type of image reconstruction is utilized to see where in the skeleton bone mineral density loss has occurred.

FIGS. 9A and 9B are X-ray images of the femurs (sagittal cut) from wild type (FIG. 9A) and stable PAI-1 transgenic (FIG. 9B) mice showing the reduction in the cortical thickness of the femur of PAI-1 transgenic mice.

FIGS. 10A-10J depict Masson's trichrome stains of representative livers from wild type and treated mice.

FIGS. 10A-10C depict Masson's trichrome stained livers from L-NAME-treated wild type mice. FIGS. 10D-10F depict Masson's trichrome stained livers from L-NAME+tiplaxtinin (PAI-039)-treated wild type mice. FIGS. 10G-10I depict Masson's trichrome stained livers from L-NAME-treated PAI-1^{-/-} mice. Each section was taken after 6 weeks of L-NAME treatment, and the magnification of each section is indicated to the far left. Arrows illustrate the extent of venous thrombi in wild type mice in FIG. 10A. FIG. 10J presents the results of calculating the percent occluded luminal area in all three treatment groups (p<0.001 for L-NAME-treated wild type mice vs. L-NAME-treated PAI-1^{-/-} mice, and p<0.001 for L-NAME-treated wild type mice vs. wild type mice treated with L-NAME and tiplaxtinin, by ANOVA).

FIGS. 11A and 11B depict the results of assaying blood from various treatment groups for the presence of total bilirubin, AST, and active PAI-1.

FIG. 11A depicts the results of assaying total serum bilirubin (top panel) and AST (bottom panel), and shows that levels of each were elevated in wild type mice receiving L-NAME, but not in PAI-1^{-/-} mice or in wild type mice receiving tiplaxtinin (PAI-039). FIG. 11B depicts the results of assaying PAI-1 activity, and shows that tiplaxtinin (PAI-039) reduced plasma PAI-1 activity in wild type mice receiving L-NAME.

### BRIEF DESCRIPTION OF THE SEQUENCE LISTING

SEQ ID NOs: 1 and 2 are the nucleotide (GENBANK® Accession No. X16383) and encoded amino acid sequences, respectively, of a bovine PAI-1 gene product.

SEQ ID NOs: 3 and 4 are the nucleotide (GENBANK® Accession No. M16006) and encoded amino acid sequences, respectively, of a human plasminogen activator inhibitor-1 (PAI-1) mRNA.

SEQ ID NOs: 5 and 6 are the nucleotide (GENBANK® Accession No. X04744) and encoded amino acid sequences, respectively, of a human mRNA for plasminogen activator inhibitor (PAI-1).

SEQ ID NOs: 7 and 8 are the nucleotide (GENBANK® Accession No. X58541) and encoded amino acid sequences, respectively, of a mink mRNA for plasminogen activator inhibitor type 1.

SEQ ID NOs: 9 and 10 are the nucleotide (GENBANK® Accession No. NM_{—}008871) and encoded amino acid sequences, respectively, of a mouse PAI-1 gene product.

SEQ ID NOs: 11 and 12 are the nucleotide (GENBANK® Accession No. M33960) and encoded amino acid sequences, respectively, of a mouse PAI-1 gene product.

SEQ ID NOs: 13 and 14 are the nucleotide (GENBANK® Accession No. M24067) and encoded amino acid sequences, respectively, of a rat PAI-1 gene product.

SEQ ID NOs: 15 and 16 are the nucleotide sequences of the oligonucleotide primers used to amplify a 260 basepair polyadenylation signal from SV40.

### DETAILED DESCRIPTION

The presently disclosed subject matter pertains in part to the pathological consequences of impaired activation of plasminogen system by chronic overexpression of active human PAI-1 under the control of mPPET-1 promoter. Disclosed herein is the remarkable phenotypic alterations exhibited by newly engineered lines of transgenic mice that overexpress a stable variant of human PAI-1 under the control of the mPPET-1 promoter. These transgenic animals manifest time-dependent alopecia areata, hepatosplenomegaly, and evidence of extramedullary hematopoiesis. Microscopic examination of the spleen and liver reveals that the enlargement and architectural disruption observed in both organs are due to extracellular matrix and amyloid deposition, and in the spleen, also due to the presence of hematopoietic precursors (including megakaryocytes). These animals also exhibit glomerulosclerosis and renal fibrosis. Taken together, these findings indicate that PAI-1 influences a broad spectrum of processes involving cellular migration and matrix proteolysis, which findings are useful for determining the pathogenesis of and providing for treatment of disorders as complex as systemic amyloidosis and myelofibrosis, and as pervasive as pattern baldness.

Thus, the presently disclosed subject matter provides, in some embodiments, therapeutic methods for treating vascular thrombic disorders, asthma, chronic obstructive pulmonary disease, Alzheimer's Disease, myelofibrosis, wasting disorders characterized by weight loss (e.g., anorexia, AIDS, etc.), systemic amyloidosis, alopecia, male pattern baldness, glomerulosclerosis, keloids, apocrine cysts, acne, atherosclerosis, aging, a wound, veno-occlusive disease (VOD), obesity, non-alcoholic steatohepatitis (NASH), osteoporosis, osteopenia, polycystic ovarian syndrome (PCOS), and combinations thereof, in subjects in need of such treatment.

In another embodiment, the presently disclosed subject matter provides a transgenic non-human vertebrate animal having a PAI-1 gene incorporated into its genome. In some embodiments, the incorporation of the PAI-1 gene results in the overexpression of PAI-1 in the animal. In some embodiments, the animal is a transgenic mouse. Also provided is a construct comprising a PAI-1 gene encoding a biologically active PAI-1 polypeptide and a vector. In some embodiments, the construct is employed in the production of the transgenic non-human animal of the presently disclosed subject matter.

In some embodiments, the presently disclosed subject matter provides a method of employing such transgenic animals to test candidate compositions to determine if they have PAI-1 inhibition activity.

Before the present therapeutic methods as well as the present transgenic animals and uses thereof are described, it is to be understood that the presently disclosed subject matter is not limited to the particular methodology, protocols, cell lines, animal species or genera, constructs, and reagents described, as such may, of course, vary. It is also to be understood that the terminology used herein is for the purpose of describing representative embodiments only, and is not intended to limit the scope of the presently disclosed subject matter.

It must be noted that as used herein and in the appended claims, the singular forms “a”, “an”, and “the” include plural referents unless the context clearly dictates otherwise. Thus, for example, reference to “a construct” includes a plurality of such constructs and reference to “the PAI-1-encoding nucleic acid” includes reference to one or more PAI-1-encoding nucleic acids and to equivalents thereof known to those skilled in the art, and so forth.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

All publications mentioned herein are incorporated herein by reference for the purpose of describing and disclosing, for example, the cell lines, constructs, and methodologies that are described in the publications, which might be used in connection with the presently described subject matter. The publications discussed above and throughout the text are provided solely for their disclosure prior to the filing date of the present application. Nothing herein is to be construed as an admission that the inventors are not entitled to antedate such disclosure by virtue of prior invention.

I. Definitions

“Antibodies” refers to whole antibodies and antibody fragments or molecules including antibody fragments, including, but not limited to single chain antibodies, humanized antibodies, and Fab, F(ab′)₂, Vₕ, Vₗ, Fd, and single or double chain Fv fragments.

The term “medical condition associated with PAI-1 biological activity” can include any medical condition associated with PAI-1 biological activity. In some embodiments, this term includes, but is not limited to a medical condition selected from the group consisting of vascular thrombic disorders, asthma, chronic obstructive pulmonary disease (COPD), pulmonary fibrosis, alopecia, undesired weight loss (such as associated with anorexia or with a disease characterized by wasting—e.g., AIDS), Alzheimer's Disease, nephrosclerosis (including, but not limited to glomerulosclerosis), arteriosclerosis (e.g., atherosclerosis), systemic amyloidosis, myelofibrosis, pattern baldness (male or female), keloids, apocrine cysts, acne, aging, a wound, veno-occlusive disease (VOD), obesity, non-alcoholic steatohepatitis (NASH), osteoporosis, osteopenia, polycystic ovarian syndrome (PCOS), and combinations thereof. Glomerulosclerosis includes, but is not limited to diabetic and non-diabetic glomerulosclerosis.

As used herein, the term “veno-occlusive disease (VOD)” refers to venous thrombosis (for example, of the hepatic and/or portal veins) including, but not limited to that seen as a consequence of high doses of chemotherapy and/or radiation, viral infection, or subsequent to bone marrow transplantation. VOD can be diagnosed using standard clinical criteria including, but not limited to excessive bilirubin (>2 mg/dL in humans), hepatomegaly, pain in the right upper quadrant of liver origin, and unexplained weight gain.

As used herein, the term “obesity” is used in its ordinary sense and refers to a condition where a subject is characterized as being overweight (i.e. has an excess of body fat) to an extent sufficient to cause associated clinical disorders including, but not limited to diabetes, hypertension, osteoarthritis, etc. One clinical finding often associated with obesity is the infiltration of the liver with fatty deposits, which in some individuals is associated with progressive liver fibrosis, hepatic failure, and even hepatocellular carcinoma. Non-alcoholic fatty liver disease (also referred to herein as “non-alcoholic steatohepatitis” (NASH)) brought on by obesity can lead to cirrhosis, hepatic failure, and other complications.

VOD, obesity, and NASH are exemplary diseases that can potentially be treated using a hepatoprotective approach. As used herein, the term “hepatoprotection”, and grammatical variants thereof, refers to a treatment designed to protect the liver and associated tissues (for example, the hepatic vein and the portal vein) from damage. In some embodiments, hepatoprotection comprises modulating the expression or biological activity of a PAI-1 gene product (for example, by administering a PAI-1 inhibitor to a subject for which hepatoprotection is desirable).

As used herein, the terms “osteoporosis” and “osteopenia” refer to disorders where bones become less dense and more fragile. Osteopenia and osteoporosis reflect different levels of severity of reduced bone density. While osteoporosis is a common disorder that afflicts many postmenopausal women and is a common cause of morbidity and mortality in the aged population, bone density loss can occur by mechanisms that are not associated with chronological aging. Current therapies to prevent and treat osteoporosis include hormonal therapies and calcium supplementation. Other disorders that can lead to osteopenia and osteoporosis include, but are not limited to long term use of certain drugs, (for example, prednisone, heparin, and some anti-seizure medications), impaired gastrointestinal absorption, hyperparathyroidism, alcohol use, severe liver disease, and kidney failure.

As used herein, the phrase “polycystic ovarian disease (PCOS)” refers to a clinical finding wherein ovarian function is abnormal leading to the accumulation of multiple follicles in the ovaries without subsequent ovulation. The ovaries of PCOS subjects secrete higher levels of the hormones testosterone and estrogen, which can result in irregular or no menses, excess body hair growth, and often obesity, diabetes, and hypertension. PCOS has been associated with insulin resistance.

The term “phenomena associated with PAI-1 biological activity” can include any phenomena associated with PAI-1 biological activity, including those observed in a medical condition associated with PAI-1 biological activity (for example, those medical conditions associated with PAI-1 biological activity disclosed hereinabove, and animal models thereof). Representative phenomena include, but are not limited to hair loss, hepatosplenomegaly, extramedullary hematopoiesis, systemic amyloid deposition, cerebral amyloid deposition, and combinations thereof.

The term “aging” is meant to include all physiological effects of the process of aging, including effects on brain and mental function as well as physical appearance and condition. By way of additional example, “skin aging” includes skin atrophy and means the thinning and/or general degradation of the dermis caused by free radical damage that is often characterized by an alteration and degeneration of collagen and/or elastin. In epidermis, markers of degeneration include lipofuscin granules and loss of rete pegs. Skin aging can be caused by either intrinsic or extrinsic factors such as natural chronoaging, photodamage, burns, or chemical damage.

The term “transgene” is used herein to describe genetic material that has been or is about to be artificially inserted into the genome of a warm-blooded vertebrate animal, particularly a cell of a living animal.

By “transformation” is meant a permanent or transient genetic change, in some embodiments a permanent genetic change, induced in a cell following incorporation of new DNA (i.e., DNA exogenous to the cell). Where the cell is a mammalian cell, a permanent genetic change is generally achieved by introduction of the DNA into the genome of the cell.

By “transgenic animal” is meant a non-human animal, usually a mammal (e.g., mouse, rat, rabbit, hamster, etc.), having a non-endogenous (i.e., heterologous) nucleic acid sequence present as an extrachromosomal element in a portion of its cells or stably integrated into its germ line DNA (i.e., in the genomic sequence of most or all of its cells). A heterologous nucleic acid is introduced into the germ line of such transgenic animals by genetic manipulation of, for example, embryos or embryonic stem cells of the host animal.

A “knock-out” of a gene means an alteration in the sequence of the gene that results in a decrease of function of the target gene, in some embodiments such that target gene expression is undetectable or insignificant. A knockout of an endogenous PAI-1 gene means that function of the PAI-1 gene has been substantially decreased so that expression is not detectable or only present at insignificant levels. As such, an animal (for example, a mouse) that has one or both PAI-1 genes knocked out in its genome is referred to herein as a “PAI-1 deficient” animal to indicate that the level of PAI-1 expressed in this animal is less than that seen in a wild type animal of the same species. “Knock-out” transgenics can be transgenic animals having a heterozygous knockout of the PAI-1 gene or a homozygous knockout of the PAI-1 gene. “Knock-outs” also include “conditional” knock-outs, wherein “conditional” indicates that the alteration of the target gene can occur upon, for example, exposure of the animal to a substance that promotes target gene alteration, introduction of an enzyme that promotes recombination at the target gene site (e.g., Cre in the Cre-lox system), or other method for directing the target gene alteration postnatally.

A “knock-in” of a target gene means an alteration in a host cell genome that results in altered expression (e.g., increased (including ectopic) expression) of the target gene, for example, by introduction of an additional copy of the target gene or by operatively inserting a regulatory sequence that provides for enhanced expression of an endogenous copy of the target gene.

“Knock-in” transgenics of interest for the presently disclosed subject matter can be transgenic animals having a knock-in of the animal's endogenous PAI-1. Such transgenics can be, for example, heterozygous for a knock-in of the PAI-1 gene or homozygous for a knock-in of the PAI-1 gene. “Knock-ins” also encompass “conditional” knock-ins, with “conditional” being as defined above.

By “construct” is meant a recombinant nucleic acid, generally recombinant DNA, which has been generated for the purpose of the expression of a specific nucleotide sequence(s), or is to be used in the construction of other recombinant nucleotide sequences.

By “operatively inserted” is meant that a nucleotide sequence of interest is positioned adjacent a nucleotide sequence that directs transcription and translation of the introduced nucleotide sequence of interest (i.e., facilitates the production of, e.g., a polypeptide encoded by a PAI-1 sequence).

By “operatively linked” is meant that a DNA sequence and a regulatory sequence(s) are connected in such a way as to permit gene expression when the appropriate molecules (e.g., transcriptional activator proteins) are bound to the regulatory sequence(s).

The term “subject” as used herein refers to any invertebrate or vertebrate species. The methods of the presently disclosed subject matter are particularly useful in the treatment of warm-blooded vertebrates. Thus, in a representative embodiment, the presently disclosed subject matter concerns mammals and birds.

The term “about”, as used herein when referring to a measurable value such as an amount of weight, time, dose, etc. is meant to encompass variations of in some embodiments ±20% or ±10%, in some embodiments ±5%, in some embodiments ±1%, and in some embodiments ±0.1% from the specified amount, as such variations are appropriate to perform the disclosed methods.

II. Therapeutic Methods

A method of treating a warm-blooded vertebrate animal having a medical condition in need of treatment with a composition that exhibits PAI-1 inhibition activity is provided in accordance with the presently disclosed subject matter. In some embodiments, the method comprises administering a treatment effective amount of the composition to a warm-blooded animal having a medical condition selected from one group consisting of alopecia, undesired weight loss, Alzheimer's Disease, systemic amyloidosis, myelofibrosis, pattern baldness, nephrosclerosis (including but not limited to glomerulosclerosis), arteriosclerosis (such as atherosclerosis), systemic amyloidosis, myelofibrosis, male pattern baldness, keloids, apocrine cysts, acne, aging, a wound, veno-occlusive disease (VOD), obesity, non-alcoholic steatohepatitis (NASH), osteoporosis, osteopenia, polycystic ovarian syndrome (PCOS), and combinations thereof, and observing an improvement in the medical condition in the warm-blooded animal having the medical condition. Pharmacological inhibition of PAI-1 also protects against Ang II-induced aortic remodeling. Thus, although it is not applicants' desire to be bound by any particular theory of operation, the observation of an improvement in the medical condition is believed to be indicative of inhibition activity of PAI-1.

Also provided is a method of hepatoprotection. In some embodiments, the method comprises: (a) administering a treatment effective amount of a composition that exhibits PAI-1 inhibition activity to a warm-blooded vertebrate animal in need of hepatoprotection and having a medical condition associated with PAI-1 biological activity; and (b) observing an improvement in the medical condition indicative of inhibition activity of PAI-1 in the warm-blooded animal having the medical condition.

Animals so treated can be warm-blooded vertebrates, for instance, mammals and birds. More particularly, the animal can be selected from the group consisting of rodent, swine, bird, ruminant, and primate. Even more particularly, the animal can be selected from the group consisting of a mouse, a rat, a pig, a guinea pig, poultry, an emu, an ostrich, a goat, a cow, a sheep, and a rabbit. Most particularly, the animal can be a primate, such as an ape, a monkey, a lemur, a tarsier, a marmoset, or a human.

Thus, provided is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered or kept in zoos, as well as fowl, and more particularly domesticated fowl, e.g., poultry, such as turkeys, chickens, ducks, geese, guinea fowl, and the like, as they are also of economic importance to humans. Thus, provided is the treatment of livestock, including, but not limited to domesticated swine (pigs and hogs), ruminants, horses, poultry, and the like.

The medical condition can include, but is not limited to a medical condition selected from the group consisting of alopecia, undesired weight loss, Alzheimer's Disease, systemic amyloidosis, myelofibrosis, pattern baldness, veno-occlusive disease (VOD), obesity, non-alcoholic steatohepatitis (NASH), osteoporosis, osteopenia, polycystic ovarian syndrome (PCOS), and combinations thereof.

II.A. PAI-1 Modulators

PAI-1 modulators are used in the presently disclosed methods for modulating PAI-1 activity in cells and tissues. Thus, as used herein, the terms “modulate”, “modulating”, and “modulator” are meant to be construed to encompass inhibiting, blocking, promoting, stimulating, agonising, antagonizing, or otherwise affecting PAI-1 activity in cells and tissues. PAI-1 modulators also include substances that inhibit or promote expression of a PAI-1 encoding nucleic acid segment.

In some embodiments, a PAI-1 activity inhibiting composition is employed in accordance with the presently disclosed subject matter. The terms “composition exhibiting PAI-1 inhibition activity”, “PAI-1 inhibitor” or “PAI-1 inhibiting composition” are used interchangeably and are meant to refer to a substance that acts by inhibiting, blocking, antagonizing, down regulating, or otherwise reducing PAI-1 activity in cells and tissues. These terms also encompass substances that inhibit expression of a PAI-1 encoding nucleic acid segment, e.g. an anti-sense oligonucleotide or small molecule that blocks the promoter of the PAI-1 gene.

Representative PAI-inhibitors are disclosed in U.S. Pat. No. 5,980,938 to Berg et al. (assignee: Eli Lilly and Co.), which discloses methods of inhibiting PAI-1 using benzopyran compounds. Butadiene derivatives having PAI-1 inhibitory activity and a process for preparing the same are disclosed in the U.S. Pat. No. 6,248,743 to Ohtani et al. (assignee: Tanabe Seiyaku Co.). PCT International Publication No. WO 01/51085 by Demissie-Sanders et al. (assignee: Tanox Inc.) discloses PAI-1 antagonists and their use in the treatment of asthma and chronic obstructive pulmonary disease. Representative PAI-1 inhibitors also include peptide therapeutic agents, such as those disclosed in U.S. Pat. No. 5,639,726 to Lawrence et al. (co-assignees: The Regents of the University of Michigan and the Henry Ford Health System), which discloses peptides that decrease the half-life of active PAI-1.

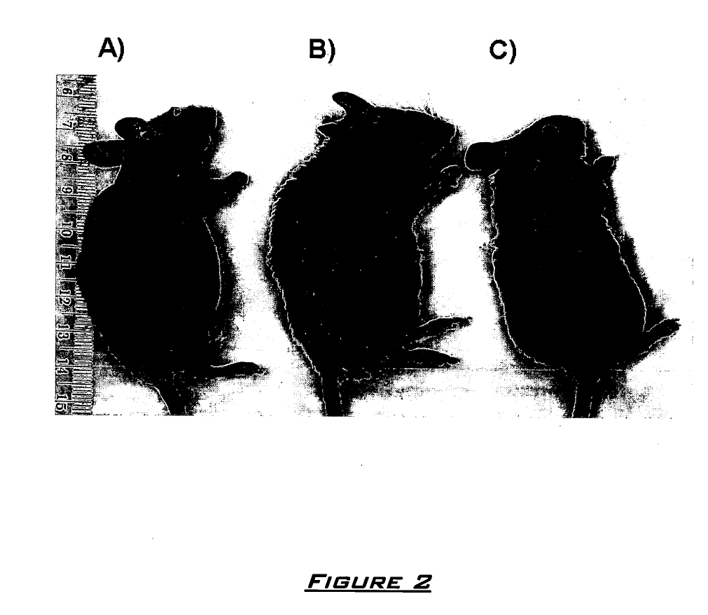

In some embodiments, a composition exhibiting PAI-1 inhibition activity comprises tiplaxtinin (PAI-039; 2-{1-Benzyl-5-[4-(trifluoromethoxy)phenyl]-1H-indol-3-yl}-2-oxoacetic acid) or a pharmaceutically acceptable salt thereof. Tiplaxtinin can be prepared via the process set forth in FIG. 2 of Elokdah et al., 2004 (the disclosure of which is incorporated herein by reference in its entirety), and has the following structure:

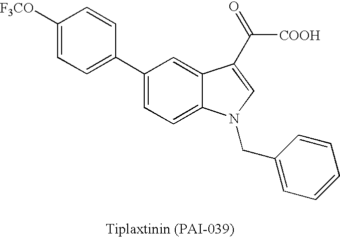

### 2-{1-Benzyl-5-[4-(trifluoromethoxy)phenyl]-1H-indol-3-yl}-2-oxoacetic acid

Antagonists to PAI-1 can be used in the treatment of the above-noted medical conditions associated with PAI-1 biological activity. Antagonists can be antibodies, peptides, proteins, nucleic acids, small organic molecules, or polymers. In some embodiments, the antagonist is an antibody. The antibody can be a monoclonal or polyclonal antibody. The antibody can be chemically linked to another organic or biomolecule. Monoclonal and polyclonal antibodies can be made by any method generally known to those of ordinary skill in the art. For example, U.S. Pat. No. 5,422,245 to Nielsen et al. (assignee: Fonden Til Fremme AF Eksperimental Cancerforskning of Copenhagen, Denmark) describes the production of monoclonal antibodies to plasminogen activator inhibitor.

Peptides, proteins, nucleic acids, small organic molecules, and polymers can be identified by combinatorial methods.

Known PAI-1 antagonists can be used, including, for example, spironolactone, imidapril, angiotensin converting enzyme inhibitors (ACEI, captopril, or enalapril), angiotensin II receptor antagonist (AIIRA), or defibrotide (a polydeoxyribonucleotide).

A PAI-1 inhibitor or antagonist is in some embodiments administered at a therapeutically effective dose or concentration. Representative concentrations of the inhibitor or antagonists include, but are not limited to less than about 10 μM, about 1 μM, about 0.1 μM, about 0.01 μM, about 0.001 μM, or about 0.0001 μM.

The therapeutic methods of the presently disclosed subject matter are also directed towards the use of compounds that change the concentration of upstream regulators or downstream effector molecules of PAI-1 and/or in treating or preventing the above-listed medical conditions associated with PAI-1. In some embodiments, the methods can comprise selecting a warm-blooded vertebrate subject diagnosed with a medical condition associated with PAI-1 biological activity, and administering to the warm-blooded vertebrate subject one or more compounds. Representative compounds can comprise urokinase, tissue plasminogen activator, vitronectin, plasminogen, plasmin, matrix metalloproteinases, or tissue inhibitors of metalloproteinases. Representative concentrations for the compound include, but are not limited to less than about 100 μM, about 10 μM, about 1 μM, about 0.1 μM, about 0.01 μM, about 0.001 μM, and about 0.0001 μM.

An additional embodiment of the presently disclosed subject matter is directed towards a method for the prevention of a medical condition associated with PAI-1 biological activity. The method can comprise selecting a warm-blooded vertebrate subject in which the prevention of a medical condition associated with the biological activity of PAI-1 is desired and administering to the warm-blooded vertebrate subject a PAI-1 inhibiting composition in an amount sufficient to reduce the occurrence or effects of the medical condition associated with PAI-1 biological activity relative to a warm-blooded vertebrate subject that did not receive such administration. The concentration of the PAI-1 inhibiting composition is in some embodiments less than about 100 μM, in some embodiments less than about 10 μM, in some embodiments less than about 1 μM, in some embodiments less than about 0.1 μM, in some embodiments less than about 0.01 μM, in some embodiments less than about 0.001 μM, and in some embodiments less than about 0.0001 μM.

II.B. Formulation of Therapeutic Compositions

The PAI-1 biological activity modulating substances, gene therapy vectors, and substances that inhibit or promote expression of a PAI-1 encoding nucleic acid segment can be adapted for administration as a pharmaceutical composition. Additional formulation and dose preparation techniques have been described in the art (see e.g., those described in U.S. Pat. No. 5,326,902 issued to Seipp et al. on Jul. 5, 1994; U.S. Pat. No. 5,234,933 issued to Marnett et al. on Aug. 10, 1993; and PCT International Publication Number WO 93/25521 of Johnson et al. published Dec. 23, 1993, the entire contents of each of which are herein incorporated by reference).

For therapeutic applications, a treatment effective amount of a composition of the presently disclosed subject matter is administered to a subject. A “treatment effective amount” is an amount of the therapeutic composition sufficient to produce a measurable biological response, such as but not limited to a reduction in PAI-1 biological activity. Actual dosage levels of active ingredients in a therapeutic composition of the presently disclosed subject matter can be varied so as to administer an amount of the active compound(s) that is effective to achieve the desired therapeutic response for a particular subject. The selected dosage level will depend upon a variety of factors including, but not limited to the activity of the therapeutic composition, the formulation, the route of administration, combinations with other drugs or treatments, and the physical condition and prior medical history of the subject being treated. In some embodiments, a minimal dose is administered, and the dose is escalated in the absence of dose-limiting toxicity. The determination and adjustment of a therapeutically effective dose, as well as evaluation of when and how to make such adjustments, are well known to those of ordinary skill in the art of medicine.

For the purposes described above, the identified substances can normally be administered systemically or partially, usually by oral or parenteral administration. The doses to be administered are determined depending upon age, body weight, symptom, the desired therapeutic effect, the route of administration, and the duration of the treatment, etc.; one of skill in the art of therapeutic treatment will recognize appropriate procedures and techniques for determining the appropriate dosage regimen for effective therapy. Various compositions and forms of administration are contemplated and are generally known in the art. Other compositions for administration include liquids for external use, and endermic liniments (ointment, etc.), suppositories, and pessaries that comprise one or more of the active substance(s) and can be prepared by known methods.

Thus, the presently disclosed subject matter provides pharmaceutical compositions comprising in some embodiments a polypeptide, polynucleotide, antibody or fragment thereof, small molecule, or compound of the presently disclosed subject matter, and a physiologically acceptable carrier. In some embodiments, a pharmaceutical composition comprises a compound discovered via the screening methods described herein.

A composition of the presently disclosed subject matter is typically administered parenterally in dosage unit formulations containing standard, well-known nontoxic physiologically acceptable carriers, adjuvants, and vehicles as desired. The term “parenteral” as used herein includes, but is not limited to intravenous, intramuscular, intra-arterial injection, or infusion techniques.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that can be employed are water, Ringer's solution, and isotonic sodium chloride solution. In addition, sterile, fixed oils are conventionally employed as solvents or suspending media. For this purpose, any bland fixed oil can be employed including synthetic mono- or diglycerides. In addition, fatty acids such as oleic acid find use in the preparation of injectables.

Representative, non-limiting carriers include neutral saline solutions buffered with phosphate, lactate, Tris, and the like. Of course, one purifies the vector sufficiently to render it essentially free of undesirable contaminants, such as defective interfering adenovirus particles or endotoxins and other pyrogens, such that it does not cause any untoward reactions in the individual receiving the vector construct. An exemplary approach for purifying the vector involves the use of buoyant density gradients, such as cesium chloride gradient centrifugation.

A transfected cell can also serve as a carrier. By way of example, a liver cell can be removed from an organism, transfected with a polynucleotide of the presently disclosed subject matter using methods set forth above, and then the transfected cell returned to the organism (e.g., injected intra-vascularly).

III. Transgenic Non-Human Animals

The term “transgene” is used herein to describe genetic material that has been or is about to be artificially inserted into the genome of a mammalian cell, particularly a mammalian cell of a living animal. The transgene is used to transform a cell, meaning that a permanent or transient genetic change, in some embodiments a permanent genetic change, is induced in a cell following incorporation of exogenous DNA. A permanent genetic change is generally achieved by introduction of the DNA into the genome of the cell.

Vectors for stable integration include plasmids, retroviruses and other animal viruses, bacterial artificial chromosomes (BACs), yeast artificial chromosomes (YACs), cosmids, and the like. The term “vector”, as used herein, refers to a DNA molecule having sequences that enable its replication in a compatible host cell. A vector also includes nucleotide sequences to permit ligation of nucleotide sequences within the vector, wherein such nucleotide sequences are also replicated in a compatible host cell. A vector can also mediate recombinant production of a PAI-1 polypeptide, as described further herein below. Representative vectors include, but are not limited to p5.9.

Useful animals should be warm-blooded non-human vertebrates, for instance, mammals and birds. More particularly, the animal can be selected from the group consisting of rodent, swine, bird, ruminant, and primate. Even more particularly, the animal can be selected from the group consisting of a mouse, a rat, a pig, a guinea pig, poultry, an emu, an ostrich, a goat, a cow, a sheep, and a rabbit. Of interest are transgenic mammals, for example cows, pigs, goats, horses, etc., and particularly rodents, for example rats, mice, etc. In some embodiments, the transgenic animals are mice.

Transgenic animals comprise an exogenous nucleic acid sequence present as an extrachromosomal element or stably integrated in all or a portion of its cells, especially in germ cells. Unless otherwise indicated, it will be assumed that a transgenic animal comprises stable changes to the germline sequence. During the initial construction of the animal, “chimeras” or “chimeric animals” are generated, in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

The exogenous gene is usually either from a different species than the animal host or is otherwise altered in its coding or non-coding sequence. The introduced gene can be a wild type gene, naturally occurring polymorphism, or a genetically manipulated sequence, for example having deletions, substitutions, or insertions in the coding or non-coding regions. Where the introduced gene is a coding sequence, it is usually operatively linked to a promoter, which can be constitutive or inducible, and other regulatory sequences required for expression in the host animal. By “operatively linked” is meant that a DNA sequence and a regulatory sequence(s) are connected in such a way as to permit gene expression when the appropriate molecules, e.g. transcriptional activator proteins, are bound to the regulatory sequence(s).

In general, the transgenic animals of the presently disclosed subject matter comprise genetic alterations to provide for expression of a biologically active PAI-1 polypeptide (Sprengers and Kluft, 1987), and/or expression of a desired biologically active PAI-1 sequence (e.g., human PAI-1; Rosenberg and Aird, 1999). In some embodiments, the introduced sequences provide for high-level expression of PAI-1 so that overexpression of the PAI-1 gene is conferred in the transgenic animal. Thus, in some embodiments, the PAI-1 transgene is overexpressed in the host animal; that is, the transgene provides for increased levels of PAI-1 production relative to wild type, e.g., more particularly a level of PAI-1 expression to facilitate onset of a medical condition associated with PAI-1 biological activity and/or the observation of phenomena associated with PAI-1 biological activity.

The transgenic animals of the presently disclosed subject matter can comprise other genetic alterations in addition to the presence of the PAI-1-encoding sequence. For example, the host's genome can be altered to affect the function of endogenous genes (e.g., an endogenous PAI-1 gene), contain marker genes, or other genetic alterations consistent with the goals of the presently disclosed subject matter.

III.A. Knockouts and Knockins

Although not necessary to the operability of the presently disclosed subject matter, the transgenic animals described herein can also comprise alterations to endogenous genes in addition to (or alternatively for PAI-1), to the genetic alterations described above. For example, the host animals can be either “knockouts” and/or “knockins” for a target gene(s) as is consistent with the goals of the presently disclosed subject matter (e.g., the host animal's endogenous PAI-1 can be “knocked out” and/or the endogenous PAI-1 gene “knocked in”). Knockouts have a partial or complete loss of function in one or both alleles of an endogenous gene of interest (e.g., PAI-1). As a result, these animals are referred to herein as “deficient” for that gene or its product. For example, a mouse that has one or both PAI-1 alleles knocked out is referred to herein as a “PAI-1 deficient” mouse. In certain contexts, the term “PAI-1 deficient mouse” refers only to a mouse that is homozygous for the knocked out allele (i.e. whose genome does not contain a functional PAI-1 gene at all). Knockins have an introduced transgene with altered genetic sequence and/or function from the endogenous gene. The two can be combined, for example, such that the naturally occurring gene is disabled, and an altered form introduced. For example, it can be desirable to knock out the host animal's endogenous PAI-1 gene, while introducing an exogenous PAI-1 gene (e.g., a human PAI-1 gene).

In some embodiments, the target gene expression is undetectable or insignificant in a knockout animal. For example, a knockout of a PAI-1 gene means that function of the PAI-1 has been substantially decreased so that expression is not detectable or only present at insignificant levels. This can be achieved by a variety of mechanisms, including introduction of a disruption of the coding sequence, e.g. insertion of one or more stop codons, insertion of a DNA fragment, etc., deletion of coding sequence, substitution of stop codons for coding sequence, etc. In some cases the exogenous transgene sequences are ultimately deleted from the genome, leaving a net change to the native sequence. Different approaches can also be used to achieve the “knockout”. A chromosomal deletion of all or part of the native gene can be introduced, including deletions of the non-coding regions, particularly the promoter region, 3′ regulatory sequences, enhancers, or deletions of a gene that activates expression of endogenous PAI-1 genes. A functional knockout can also be achieved by the introduction of an anti-sense construct that blocks expression of the native genes (see e.g., Li and Cohen, 1996). “Knockouts” also include conditional knockouts, for example where alteration of the target gene occurs upon exposure of the animal to a substance that promotes target gene alteration, introduction of an enzyme that promotes recombination at the target gene site (e.g., Cre in the Cre-lox system), or other method for directing the expression of the target gene alteration postnatally.

It should be noted that while a PAI-1 and/or host PAI-1 gene can be knocked out in the transgenic animals of the presently disclosed subject matter, it is not necessary to the utility of the transgenic PAI-1 animal. Indeed, it is envisioned that PAI-1 knockout transgenic animals would primarily serve as control animals in, for example, the drug screening assays disclosed herein below.

A “knock-in” of a target gene means an alteration in a host cell genome that results in altered expression or function of a native target gene. Increased (including ectopic) or decreased expression can be achieved by introduction of an additional copy of the target gene, or by operatively inserting a regulatory sequence that provides for enhanced expression of an endogenous copy of the target gene. These changes can be constitutive or conditional (e.g., dependent on the presence of an activator or repressor). The use of knock-in technology can be combined with production of exogenous sequences to produce the transgenic animals of the presently disclosed subject matter. For example, the PAI-1 transgenic animals of the presently disclosed subject matter can contain a knock-in of the host's endogenous PAI-1-encoding sequences to provide for the desired level of PAI-1 expression, and can contain an exogenous PAI-1-encoding sequence.

III.B. Nucleic Acid Compositions

Constructs for use in the presently disclosed subject matter include any construct suitable for use in the generation of transgenic animals having the desired levels of expression of a desired PAI-1-encoding sequence. Methods for isolating and cloning a desired sequence, as well as suitable constructs for expression of a selected sequence in a host animal, are well known in the art. The construct can include sequences other than the PAI-1-encoding sequences. For example, a detectable marker such as lacZ can be included in the construct, where upregulation of expression of the encoded sequence will result in an easily detected change in phenotype.

The PAI-1-encoding construct can contain a wild type sequence encoding PAI-1 or a mutant sequence encoding PAI-1 (providing the PAI-1 sequence, when expressed in conjunction with PAI-1 in the host animal, impacts cellular migration and matrix proteolysis, which play a role in the pathogenesis and treatment of disorders as complex as systemic amyloidosis and myelofibrosis, and as pervasive as male pattern baldness). Likewise, the PAI-1-encoding construct can contain a wild type PAI-1-encoding sequence or a sequence encoding a modified PAI-1, particularly where the modification provides for a desired level of PAI-1 expression. Regardless of the precise construct used, the encoded PAI-1 can be in some embodiments a biologically active form of a PAI-1 polypeptide.

The term “PAI-1 gene” is used generically to refer to PAI-1 genes, including but not limited to homologs from rat, human, mouse, guinea pig, etc., and their alternate forms. A human PAI-1 gene is an exemplary PAI-1 gene. “PAI-1 gene” is also intended to refer to the open reading frame encoding specific polypeptides, introns, and adjacent 5′ and 3′ non-coding nucleotide sequences involved in the regulation of expression, up to about 1 kb beyond the coding region, but possibly further in either direction. The DNA sequences encoding PAI-1 can be cDNA or genomic DNA, or a fragment thereof. The genes can be introduced into an appropriate vector for extrachromosomal maintenance or for integration into the host.

The genomic sequences of particular interest comprise the nucleic acid present between the initiation codon and the stop codon, including some or all of the introns that are normally present in a native chromosome. They can further include the 3′ and 5′ untranslated regions found in the mature mRNA. They can further include specific transcriptional and translational regulatory sequences, such as promoters, enhancers, etc., including about 1 kb, but possibly more, of flanking genomic DNA at either the 5′ or 3′ end of the transcribed region. The genomic DNA can be isolated as a fragment of 100 kb or smaller; and substantially free of flanking chromosomal sequence.

The sequences of the 5′ regions of the PAI-1 gene, and further 5′ upstream sequences and 3′ downstream sequences, can be utilized for the identification and isolation of promoter elements, including enhancer-binding sites, which provide for the expression in tissues where PAI-1 is normally expressed. The tissue specific expression is useful for providing promoters that mimic the native pattern of expression. Naturally occurring polymorphisms in the promoter region are useful for determining natural variations in expression, particularly those that can be associated with disease. For example, the most significant of these is a single guanosine insertion/deletion variation (5G or 4G) in the promoter region (4G deletion polymorphism), situated 675 base pairs upstream from the transcriptional start site of the PAI-1 gene. The 4G allele is correlated with increased plasma PAI-1 levels (Dawson et al., 1993; Hermans et al., 1999; Dawson et al., 1991; Mansfield et al., 1994).

Alternatively, mutations can be introduced into the promoter region to determine the effect of altering expression in experimentally defined systems. Methods for the identification of specific DNA motifs involved in the binding of transcriptional factors are known in the art (e.g., sequence similarity to known binding motifs, gel retardation studies, etc. See also Blackwell et al., 1995; Mortlock et al., 1996; and Joulin and Richard-Foy, 1995).

The nucleic acid compositions used in the presently disclosed subject matter can encode all or a part of PAI-1 as appropriate. Fragments can be obtained of the DNA sequence by chemically synthesizing oligonucleotides in accordance with conventional methods, by restriction enzyme digestion, by PCR amplification, etc. DNA fragments will be in some embodiments at least 15 nucleotides (nt), in some embodiments at least 18 nt, and in some embodiments at least about 50 nt. Such small DNA fragments are useful as primers for PCR, hybridization screening, etc. Larger DNA fragments, (e.g., greater than 100 nt) are useful for production of the encoded polypeptide. For use in amplification reactions such as PCR, one or more pairs of primers are typically employed.

Several isoforms and homologs of PAI-1 have been isolated and cloned. Additional homologs of cloned PAI-1 and/or PAI-1 can be identified by various methods known in the art. Nucleic acids having sequence similarity can be detected by hybridization under low stringency conditions, for example, at 50° C. and 10× standard saline citrate (1×SSC is 0.9 M saline/0.09 M sodium citrate; see Sambrook and Russell, 2001, for a description of SSC buffer and exemplary hybridization conditions) and remain bound when subjected to washing at 55° C. in 1×SSC. Sequence identity can be determined by hybridization under more stringent conditions, for example, at 50° C. or higher in 0.1×SSC (9 mM saline/0.9 mM sodium citrate). By using probes, particularly labeled probes of DNA sequences, one can isolate homologous or related genes. The source of homologous genes can be any species, including, but not limited to primates, rodents, canines, felines, bovines, ovines, equines, etc.

Where desirable, the PAI-1 sequences, including flanking promoter regions and coding regions, can be mutated in various ways known in the art to generate targeted changes in the sequence of the encoded protein, splice variant production, etc. The sequence changes can be substitutions, insertions, or deletions. Deletions can include large changes, such as deletions of a domain or exon. Other modifications of interest include epitope tagging, e.g. with the FLAG system, HA, etc. For studies of subcellular localization, fusion proteins with green fluorescent proteins (GFP) can be used. Such mutated genes can be used to study structure-function relationships of PAI-1, or to alter properties of the proteins that affect their function and/or regulation. The PAI-1 encoding sequence can also be provided as a fusion protein. Methods for production of PAI-1 constructs are well known in the art (see e.g., Wyss-Coray et al., 1995).

Techniques for in vitro mutagenesis of cloned genes are known. Examples of protocols for scanning mutations can be found in Gustin et al., 1993; Barany, 1985; Colicelli et al., 1985; and Prentid et al., 1984. Methods for site specific mutagenesis can be found in Chapter 13 of Sambrook and Russell, 2001; Weiner et al., 1993; Sayers et al., 1992; Jones and Winistorfer, 1992; Barton et al., 1990; Marotti and Tomich, 1989; and Zhu, 1989.

The PAI-1 gene, and exemplary derivatives thereof suitable for use in the production of the transgenic animals of the presently disclosed subject matter, can be either genomic or cDNA, in some embodiments cDNA, and can be derived from any source, e.g., human, murine, porcine, bovine, etc. Several PAI-1 sequences have been isolated, cloned, and sequenced. Table 1 provides a list of exemplary PAI-1 sequences that can be suitable for use in the presently disclosed subject matter, as well as GENBANK® accession numbers relating to such sequences.

TABLE 1
Representative PAI-1 Nucleic Acid Sequences

| PAI-1 Sequence(s) | GENBANK ® Accession Nos. and Descriptions |
|---|---|
| Bovine PAI-1 | X16383 (SEQ ID NO: 1); bovine PAI-1 mRNA |
| Human PAI-1 | M16006 (SEQ ID NO: 3); human plasminogen |
| | activator inhibitor-1 (PAI-1) mRNA, complete |
| | coding sequence |
| | X04744 (SEQ ID NO: 5); human PAI-1 mRNA |
| | AH002922; human plasminogen activator |
| | inhibitor 1 (PAI-1) gene, exon 1 |
| Mink PAI-1 | X58541 (SEQ ID NO: 7); Mink PAI-1 mRNA |
| Mouse PAI-1 | NM_008871 (SEQ ID NO: 9); Mus musculus |
| | plasminogen activator inhibitor, type I |
| Rat PAI-1 | M33960 (SEQ ID NO: 11); Mouse |
| | plasminogen activator inhibitor (PAI-1) mRNA, |
| | complete coding sequence |
| | M24067 (SEQ ID NO: 13); Rattus |
| | norvegicus plasminogen activator inhibitor-1 |
| | (PAI-1) mRNA, complete coding sequence |

The host animals can be homozygous or heterozygous for the PAI-1-encoding sequence (for example, homozygous). The PAI-1 gene can also be operatively linked to a promoter to provide for a desired level of expression in the host animal and/or for tissue-specific expression. Expression of PAI-1 can be either constitute or inducible (for example, constitutive). In some embodiments, PAI-1 gene expression is driven by a strong promoter, for example the mouse preproendothelin-1 (mPPET-1) gene promoter.

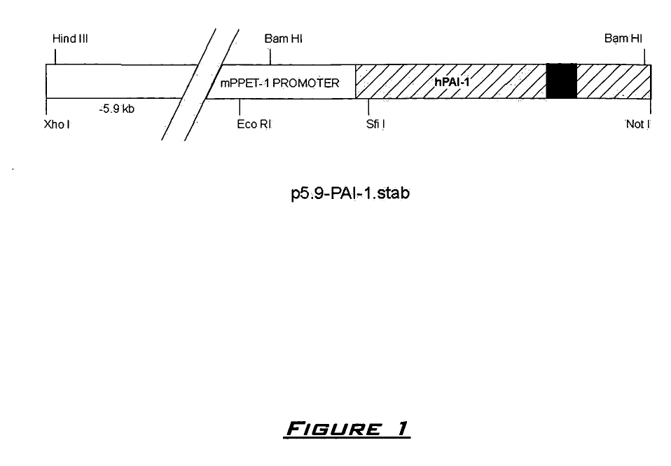

Indeed, in general terms, in an exemplary embodiment, a transgene of the presently disclosed subject matter was prepared in the following manner. The stable human PAI-1 gene was cloned into a plasmid containing the mouse preproendothelin-1 (mPPET-1) gene promoter (5.9 kb). The Xho I-Not I restriction enzyme digest fragment of p5.9-PAI-1.stab was used for microinjections to generate the transgenic mouse. See FIG. 1.

In some embodiments, PAI-1 transgenic animals overproduce biologically active PAI-1 relative to control, non-transgenic animals. For example, PAI-1 transgenic animals can exhibit PAI-1 mRNA levels in blood, skin, heart, lung, aorta, bone marrow, pancreas, kidney, brain, liver, and/or spleen that are greater than PAI-1 mRNA levels in blood, skin, heart, lung, aorta, bone marrow, pancreas, kidney, brain, liver, and/or spleen of non-transgenic animals. In some embodiments, the PAI-1 mRNA levels in blood, skin, heart, lung, aorta, pancreas, kidney, brain, liver, and/or spleen are elevated by about one- to two-fold in heterozygous PAI-1 animals, and about five-to six-fold in homozygous PAI-1 animals, relative to PAI-1 mRNA levels in non-transgenic control animals (e.g., in littermate control animals). Methods for assessment of PAI-1 mRNA levels, as well as other methods for assessing PAI-1 production and activity, are well known in the art.

III.D. Methods of Making Transgenic Animals

It is thus within the scope of the presently disclosed subject matter to prepare a transgenic non-human animal that expresses, and in some embodiments overexpresses, a PAI-1 gene. An exemplary transgenic animal is a mouse.

Techniques for the preparation of transgenic animals are known in the art. Exemplary techniques are described in U.S. Pat. No. 5,489,742 (transgenic rats); U.S. Pat. Nos. 4,736,866; 5,550,316; 5,614,396; 5,625,125; and 5,648,061 (transgenic mice); U.S. Pat. No. 5,573,933 (transgenic pigs); U.S. Pat. No. 5,162,215 (transgenic avian species) and U.S. Pat. No. 5,741,957 (transgenic bovine species), the entire contents of each of which are herein incorporated by reference.

With respect to a representative method for the preparation of a transgenic mouse, cloned recombinant or synthetic DNA sequences or DNA segments encoding a PAI-1 gene product are injected into fertilized mouse eggs. The injected eggs are implanted in pseudopregnant females and are grown to term to provide transgenic mice whose cells express a PAI-1 gene product.

DNA constructs for random integration need not include regions of homology to mediate recombination. Where homologous recombination is desired, the DNA constructs will comprise at least a portion of the target gene with the desired genetic modification, and will include regions of homology to the target locus. Conveniently, markers for positive and negative selection are typically included. Methods for generating cells having targeted gene modifications through homologous recombination are known in the art. For various techniques for transfecting mammalian cells, see Keown et al., 1990.

For embryonic stem (ES) cells, an ES cell line can be employed, or embryonic cells can be obtained freshly from a host, e.g. mouse, rat, guinea pig, etc. Such cells are grown on an appropriate fibroblast-feeder layer or grown in the presence of appropriate growth factors, such as leukemia inhibiting factor (LIF). When ES cells have been transformed, they can be used to produce transgenic animals. After transformation, the cells are plated onto a feeder layer in an appropriate medium. Cells containing the construct can be detected by employing a selective medium. After sufficient time to allow colonies to grow, they are picked and analyzed for the occurrence of homologous recombination or integration of the construct. Those colonies that are positive can then be used for embryo manipulation and blastocyst injection. Blastocysts can be obtained from 4 to 6 week old normally mated or superovulated females. The ES cells are trypsinized, and the modified cells are injected into the blastocoel of the blastocyst. After injection, the blastocysts are returned to each uterine horn of pseudopregnant females. Females are then allowed to proceed to term and the resulting litters are screened for the presence of the construct. By providing for a different phenotype of the blastocyst (i.e., wild type) and the ES cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture.

A transgenic animal of the presently disclosed subject matter can also comprise a mouse with a targeted modification of a PAI-1 gene. Mouse strains with complete or partial functional inactivation of the PAI-1 gene in all somatic cells can be generated using standard techniques of site-specific recombination in murine embryonic stem cells. See Capecchi, 1989; Thomas and Capecchi, 1990.

Alternative approaches include the use of anti-sense or ribozyme PAI-1 constructs, driven by a universal or tissue-specific promoter, to reduce levels of PAI-1 in somatic cells, thus achieving a “knock-down” of individual isoforms (Luyckx et al., 1999). The presently disclosed subject matter also provides for the generation of murine strains with conditional or inducible inactivation of the PAI-1 gene (Sauer, 1998; Ding et al., 1997).

The presently disclosed subject matter also provides mouse strains with specific “knock-in” modifications of the PAI-1 gene. These include mice with genetically and functionally relevant point mutations in the PAI-1 gene, in addition to manipulations such as the insertion of specific repeat expansions.

IV. Drug Screening Assays

A method of testing a candidate composition for PAI-1 inhibition activity is also provided in accordance with the presently disclosed subject matter. A wide variety of assays can be used for this purpose, e.g. determination of the localization of drugs after administration, immunoassays to detect amyloid deposition, and the like. Depending on the particular assay, whole animals can be used, or cells derived therefrom. Cells can be freshly isolated from an animal, or can be immortalized in culture. Cells of particular interest are derived from blood, bone marrow, skin, heart, lung, aorta, pancreas, kidney, brain, liver, and/or spleen.

In some embodiments, the method comprises obtaining a transgenic non-human warm blooded vertebrate animal having incorporated into its genome a PAI-1 gene encoding a biologically active PAI-1 polypeptide, the PAI-1 gene being present in the genome in a copy number effective to confer overexpression in the transgenic non-human animal of the PAI-1 polypeptide; administering a candidate composition to the transgenic non-human animal; and observing the transgenic non-human animal for determination of a change (for example, an ameliorating change) in the transgenic non-human animal indicative of inhibition of the activity of PAI-1.

In some embodiments, the observed change is a change in a phenomena associated with PAI-1 biological activity. The medical condition can include, but is not limited to a medical condition selected from the group consisting of hair loss, hepatosplenomegaly, extramedullary hematopoiesis, renal fibrosis, systemic amyloid deposition, vascular thrombic disorders, asthma, chronic obstructive pulmonary disease (COPD), pulmonary fibrosis, alopecia, undesired weight loss (such as associated with anorexia or with a disease characterized by wasting: e.g., AIDS), Alzheimer's Disease, nephrosclerosis (including, but not limited to glomerulosclerosis), arteriosclerosis (such as atherosclerosis), systemic amyloidosis, myelofibrosis, pattern baldness (male or female), keloids, apocrine cysts, acne, aging, a wound, veno-occlusive disease (VOD), obesity, non-alcoholic steatohepatitis (NASH), osteoporosis, osteopenia, polycystic ovarian syndrome (PCOS), and combinations thereof. Pharmacological inhibition of PAI-1 can also protects against Ang Il-induced aortic remodeling.

The transgenic animal is useful for testing candidate compositions to determine if they are effective as medicaments for treating various medical conditions by inhibiting PAI-1 expression in warm-blooded vertebrate animals having one or more of the medical conditions. For example, the transgenic animal can exhibit a medical condition, such as alopecia. Then, a candidate composition suspected of having PAI-1 inhibition activity, is administered to the animal. Next, the animal is observed to determine whether a change occurs that is indicative of inhibition of PAI-1 activity. In this instance, the hoped for ameliorating change is the growth of hair or the reduction or prevention of hair loss. If the ameliorating change does occur, then the composition is likely useful as a medicament in a method for treating an animal having a medical condition, such as alopecia.

A number of assays are known in the art for determining the effect of a drug on medical conditions and phenomena associated with PAI-1 biological activity. Some examples are provided above, although it will be understood by one of skill in the art that many other assays can also be used. In some embodiments, the subject animals themselves are used, alone or in combination with control animals. Control animals can have, for example, a wild type PAI-1 transgene that is not overexpressed, or can be PAI-1 “knockout” transgenics.

The screen using the transgenic animals of the presently disclosed subject matter can employ any phenomena associated with PAI-1 biological activity that can be readily assessed in an animal model. The screening can include assessment of phenomena including, but not limited to: analysis of molecular markers (e.g., levels of expression of PAI-1 gene products in blood, skin, heart, lung, aorta, pancreas, kidney, brain, liver, and/or spleen); and measurement of PAI-1 activity in plasma or tissues.

Thus, through use of the subject transgenic animals or cells derived therefrom, one can identify ligands or substrates that modulate medical conditions associated with PAI-1 biological activity. Of particular interest are screening assays for candidate compositions that have a low toxicity for human cells.

The term “candidate composition”, as used herein, refers to any molecule, e.g., a protein or pharmaceutical, with the capability of affecting a molecular and/or clinical phenomena associated with PAI-1 activity. Generally, pluralities of assay mixtures are run in parallel with different candidate composition concentrations to obtain a differential response to the various concentrations. Typically, one of these concentrations serves as a negative control (i.e., at zero concentration or below the level of detection).

Candidate compositions encompass numerous chemical classes, though typically they are organic molecules, in some embodiments small organic compounds having a molecular weight of more than 50 and less than about 2,500 Daltons. Candidate compositions can comprise functional groups necessary for structural interaction with proteins, particularly hydrogen bonding, and typically include at least an amine, carbonyl, hydroxyl or carboxyl group, and sometimes at least two of the functional chemical groups. The candidate compositions often comprise cyclical carbon or heterocyclic structures and/or aromatic or polyaromatic structures substituted with one or more of the above functional groups. Candidate compositions are also found among biomolecules including, but not limited to peptides, saccharides, fatty acids, steroids, purines, pyrimidines, derivatives and structural analogs thereof, and combinations thereof.

Candidate compositions can be obtained from a wide variety of sources including libraries of synthetic or natural compounds. For example, numerous approaches are available for random and directed synthesis of a wide variety of organic compounds and biomolecules, including expression of randomized oligonucleotides and oligopeptides. Alternatively, libraries of natural compounds in the form of bacterial, fungal, plant, and animal extracts are available or can be readily produced. Additionally, natural or synthetically produced libraries and compounds can be readily modified through conventional chemical, physical, and biochemical approaches, and can be used to produce combinatorial libraries. Known pharmacological agents can be subjected to directed or random chemical modifications, such as acylation, alkylation, esterification, amidification, etc., to produce structural analogs.

### EXAMPLES

The following Examples have been included to illustrate exemplary modes of the presently disclosed subject matter. Certain aspects of the following Examples are described in terms of techniques and procedures found or provided by the present inventors to work well in the practice of the presently disclosed subject matter. These Examples are exemplified through the use of standard laboratory practices of the inventors. In light of the present disclosure and the general level of skill in the art, those of skill will appreciate that the following Examples are intended to be exemplary only and that numerous changes, modifications, and alterations can be employed without departing from the spirit and scope of the presently disclosed subject matter.

### Materials and Methods Employed in Examples

Construction of Transgene and Generation of Transgenic Mice. The plasmids p5.9Luc and pET2.5 carrying a 5.9 kb and 1.4 kb upstream sequences of mouse preproendothelin-1 (mPPET-1) gene promoter respectively, were a gift from Dr. T. Quertermus. Harats et al. have shown that mPPET-1 promoter is specifically expressed in the endothelial cells of vascular wall as well as other tissues (Harats et al., 1995). As discussed above, a series of amino acid substitutions (N150H, K154T, Q301P, Q315L, and M3541) were produced that resulted in stabilization of reactive center loop of human PAI-1 in the active conformation (referred to as PAI-1.stab) and extended the T_{1/2} of the enzyme from 2.5 hrs to >145 hrs at 37° C. in vitro (Berkenpas et al., 1995).

The coding domain sequences for stable PAI-1 were amplified by PCR from the plasmid pMaPAI-1.stab using the high fidelity BIO-X-ACT™ DNA polymerase enzyme (Bioline of Springfield, N.J., United States of America), introducing Bam HI site at the 5′-end and a Bgl II site at the 3′-end, and ligating Bam HI/BgI II double-digested insert into the appropriate cloning sites of pGEM-T EASY™ vector (Promega of Madison, Wis., United States of America). Subsequently, the fragment encoding the PAI-1 signal peptide was restored by subcloning a Bam HI/Sfi I fragment from pUC18-PAI-1.wt plasmid into the same sites in this vector. The Bam HI/Spe I fragment from pGEM-PAI-1.stab and Xba I/Bam HI fragment from pGL3-BASIC™ (Promega) containing SV40 polyadenylation signal sequences were ligated into the Bgl II site of pET2.5 and the resulting plasmid was designated pET2.5-PAI-1.stab. A 4.2 kb Bam HI fragment from pET2.5-PAI-1.stab containing sequences −1.4 kb from mET-1 promoter, the PAI-1.stab gene, an SV40 polyadenylation signal, and the first exon and first intron of the mET-1 gene, was cloned into the Bam HI site of p5.9-Luc plasmid replacing the luciferase gene. The final plasmid construct was designated p5.9-PAI-1.stab (11.6 kb) and it contained 5.9 kb of the mET-1 promoter operatively linked to a human stable PAI-1 gene with signal peptide, SV40 polyadenylation signal, and the first exon and intron of the mET-1 gene. The orientation and sequences of cloned inserts in this plasmid was confirmed by DNA sequencing.

The 8.4 kb transgenic construct containing the 5.9 kb 5′ flanking promoter region-PAI-1.stab-SV40 Poly A signal-first exon and first intron of mPPET-1 was excised from p5.9-PAI-1 with Xho I and Not I enzymes and then purified from low melting agarose gel by extraction of DNA over a spin column (QIAGEN of Valencia, Calif., United States of America). Microinjection into one-cell embryos retrieved from B₆D₂ F1 hybrid mice was performed at the Vanderbilt University Transgenic/ES Cell Shared Resources. A ³²P-labeled DNA probe designed to hybridize to the SV40 Poly A signal (by REDIPRIME™ labeling kit, Amersham Pharmacia Biotech, Inc., Piscataway, N.J., United States of America) was used for Southern blot hybridization of Eco RI and Cla 1-digested genomic DNA from tail biopsies in EXPRESSHYB™ solution (Clontech, Palo Alto, Calif., United States of America) to identify transgenic founder animals.

Determination of PAI-1 Antigen. Blood samples were collected in 1.5 ml microfuge tubes containing 3.8% sodium citrate (pH 5.4) in a 1:9 ratio respectively, and blood cells were precipitated at 3000 rpm for 15 minutes at 4° C. and the supernatant was frozen at 70° C. until the time of assay. Tissue samples from mice were frozen in liquid nitrogen within 3 minutes of collection and stored at −80° C. Frozen tissues were homogenized with a polytron in TGH buffer (20 mM HEPES, pH 7.4, 50 mM NaCl, 10% glycerol and 1% Triton X-100) containing a cocktail of protease inhibitors (Roche, Indianapolis, Ind., United States of America) on ice (3 pulses of 20 seconds each with 2 minute intervals of incubation on ice). The tissue to TGH buffer ratio was 0.1 g/1.0 ml buffer. The proteins in the homogenized samples were extracted further by mixing on a tilt-board for 10 minutes at 4° C. These samples were spun in a microcentrifuge (14,000 rpm for 10 minutes) at 4° C. The supernatant was transferred to a new tube and frozen and stored at −80° C. PAI-1 antigen levels in samples were determined by using a chromogenic substrate assay kit from Biopool International of Broomfield, Colo., United States of America (Cat. #: 211000).

Immunofunctional assay of PAI-1 activity. The assay for PAI-1 activity was similar in principle to the method described in Ngo and Declerck, 1999. PAI-1/t-PA complexes were formed by incubating samples with an excess amount of t-PA and then quantitated by a sandwich ELISA method that took advantage of an anti-PAI-1 monoclonal antibody (MA-21F7) and an anti-t-PA antibody (MA-51H8). The amount of PAI-1/t-PA complexes was dependent on the amount of active PAI-1 in the sample. Recombinant PAI-1.stab protein was used to generate a standard curve where the activity was expressed in ng/ml active PAI-1.

The microtiter plates were coated with a 200 μl of 4.0 μg/ml anti-PAI-1 antibody solution diluted in 1×PBS buffer (pH 7.4) for 48 hrs at 4° C. After removing the excess antibody, the wells were treated for 2 hours at room temperature with 200 μl of PBS containing 1% bovine serum albumin (BSA). The wells were then washed with 200 μl of PBS containing 0.002% Tween 80 (PBS-Tween) and finally with PBS containing 10% mannitol and 2% saccharose. Samples to be assayed were diluted at least 1:5 in PBS-Tween containing 0.1% BSA and 5 mM EDTA and preincubated with an excess of human t-PA (final concentration 20 ng/ml, 37° C. for 25 minutes). Then, 180 μl of each sample was applied to the wells. After incubation for about 18 hours at 4° C. in a moist chamber, the wells were rinsed with PBS-Tween. The plates were filled with 170 μl aliquots of a horseradish peroxidase (HRP) conjugated monoclonal antibody (MA-51H8, directed against t-PA) diluted with PBS-Tween containing 1 mg/ml BSA, and incubated for 2 hours at room temperature. After repeated washing of the plates, the peroxidase reaction was performed by addition of 160 μl aliquots of a 0.1 M citrate/0.2 M sodium phosphate buffer, pH 5.0, containing 300 μg/ml o-phenylenediamine and 0.003% hydrogen peroxide. After 30 minutes to 1 hour at room temperature, the reaction was stopped with 50 μl of 4 M H₂SO₄ and the absorbance at 492 nm was measured.

Histological Analysis and Immunohistochemical Detection of PAI-1. Mouse tissues were fixed in 4% paraformaldehyde overnight followed by embedding in paraffin and sectioning at 5 microns. Sections were deparaffinized before performing established protocols for hemotoxylin/eosin, Masson's trichrome, and Congo Red stainings. Rabbit anti-rat PAI-1 (American Diagnostica, Greenwich, Conn., United States of America, catalog number 1062) was used for detection of stable PAI-1 antigen. The antigen retrieval was done with RETRIEVIT™ (pH 8.0) reagent (InnoGenex, Inc., San Ramon, Calif., United States of America) by microwaving the slides 4 times for 5 minutes each. After quenching the endogenous peroxidase activity in a 3% H₂O₂ solution, the sections were blocked with 10% POWERBLOCK™ solution (BioGenex, Inc., San Ramon, Calif., United States of America) which was diluted in 1×PBS buffer containing 0.1% BSA and 0.4% Triton X-100 for 15 minutes. The primary antibody, also diluted in 10% POWERBLOCK™ solution, was added to the sections and incubated at 4° C. for overnight in a humid chamber. The secondary antibody was biotinylated goat anti-rabbit IgG from BioGenex, Inc. (catalog number HK 394-9R), which was incubated with the tissue sections for 20 minutes in the humid chamber at the room temperature. The streptavidin-HRP conjugate (InnoGenex, catalog number CJ-1005-50) and the chromogenic substrates diamino-benzidine (DAB) or 3-aminoethyl carbazole (AEC) were used for visualization of immunoreactivity. The sections were counter-stained with hemotoxylin to see the cellular architecture.

RNA Isolation and RT-PCR. Mouse tissues were homogenized in RNAzol (0.1 g tissue/ml RNAzol) with a polytron. The RNA from the aqueous phase was precipitated with an equal volume of isopropanol and washed with 70% ethanol and resuspended in DEPC-treated water. One μg of total RNA was added into the Access RT-PCR (Promega) mix to detect the transcription of PAI-1.stab transgene by detecting the presence in the putative transgenic genomes of the 260 bp SV40 Poly A signal used in the transgene construction. The primers used to amplify the 260 bp SV40 Poly A signal were CTAGAGTCGGGGCGGC (SEQ ID NO: 15) for the 5′ end and CTTATCGATTTTACCACATTTGTAGAGG (SEQ ID NO: 16) for the 3′ end of the amplicon.

### Example 1

### Preparation of Transgenic Mice

PAI-1 is the major physiological inhibitor of plasminogen activation. To explore the impact of chronic overexpression of PAI-1 on vascular pathology, a strain of transgenic mice was developed in which the mice expressed a mutant, conformationally stable, human PAI-1 under the control of the murine preproendothelin-1 promoter. As depicted in FIG. 1, the stable human PAI-1 gene was cloned into a plasmid containing the mouse preproendothelin-1 (mPPET-1) gene promoter (5.9 kb). The Xho I-Not I Fragment of p5.9-PAI-1.stab was used for microinjections to generate the transgenic mouse.

Transient transfection of this plasmid into bovine aortic endothelial cells (BAEC) and rat aortic smooth muscle (RASM) cells confirmed the endothelial specificity of this promoter in vitro. Microinjection of 5 ng of PAI-1.stab transgene construct into one-cell embryos retrieved from B₆D₂ F1 hybrids produced 64 live-born pups. Two transgenic founder mice lines were identified by Southern blot hybridization and by determination of PAI-1 antigen levels in the plasma. The copy number of the PAI-1.stab transgene was twice as high in Line I as it was in Line II (quantified by PHOSPHORIMAGER™ analysis). The hemizygous animals from founder Line I and Line II had plasma PAI-1 levels of 10.7+3.1 ng/ml (n=6) and 5.5+2.7 ng/ml (n=6) respectively, with a p<0.0001 by ANOVA. Due to the higher PAI-1 levels in plasma, founder Line I was chosen for further characterization.

### Example 2

### Phenotype of Transgenic Mice

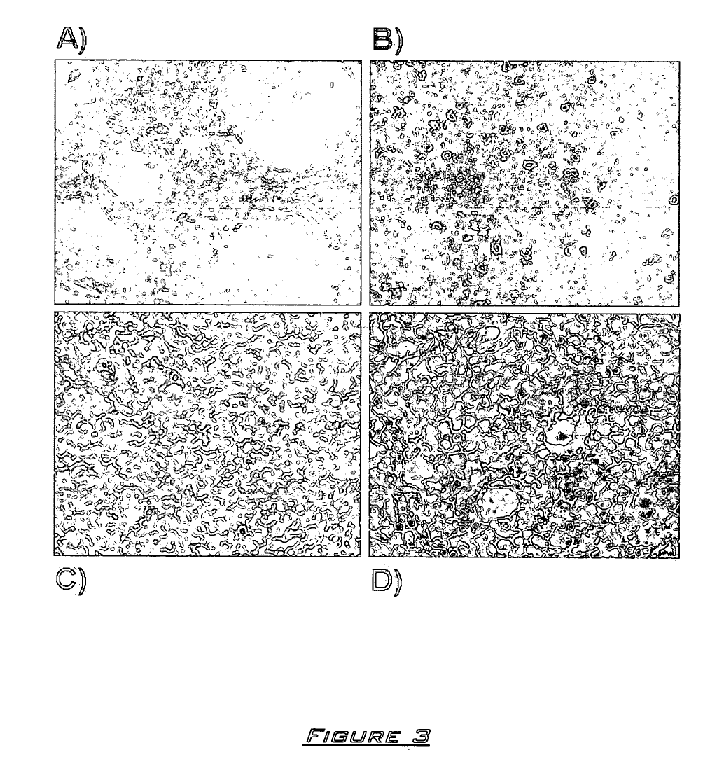

Transgenic founders and their offspring exhibited a readily detectable and permanent pattern of patchy to complete hair loss that strongly correlated with plasma PAI-1 levels and transgene copy number. PAI-1 ELISA and RT-PCR analyses detected transgene expression in skin, heart, lung, aorta, pancreas, kidney, brain, liver, and spleen, as well as in plasma. Transgenic animals also exhibited decreased intraperitoneal fat and splenomegaly (2.91 fold, n=15) compared to wild type animals. Backcrossed homozygous transgenic animals had an exaggerated phenotype including complete alopecia, absence of subcutaneous fat, hepatosplenomegaly (5.42 fold for spleen, and 1.9 fold for liver, n=6), and fibrotic lesions on the skin and face. See FIGS. 2 and 3. The massive splenomegaly was due to disruption of the normal splenic architecture by extramedullary hematopoiesis, while the hepatomegaly reflected increased matrix and amyloid deposition.

Both lines of PAI-1.stab transgenic mice initially displayed a coat with wavy hair and then a pattern of patchy to complete hair loss. Both lines also had no vibrissae when compared to their wild type littermates. In particular, homozygous animals of Line I at 6 to 8 weeks post-partum began to lose hair excessively, and eventually developed complete alopecia and unusual fibrotic lesions in the skin and face as well as apocrine cysts in the skin. These lesions occasionally became necrotic spots in the skin, where disappearance of epithelium, accumulation of neutrophils, and bacterial growth were observed. The expression of the stable PAI-1 gene resulted in striking differences in epidermal morphology of transgenic mice relative to the wild type animals. Sections from the dorsal, muzzle, and tail skin showed thickening of the epidermal layer, reduced hair follicular density, and impaired follicular keratinization pattern in transgenic mice. Microscopic analysis indicated that a disorganized keratinization and pigmentation pattern also existed in hair strands of hemizygous transgenic animals. Excessive fibrosis or connective tissue, most probably due to collagen deposits observed in transgenic skin, appeared to be constraining the hair follicles. The histochemical examination of skin sections showed signs of a reduction in subcutaneous fat relative to the wild type animal. The screening for Oil Red'O staining intensity confirmed that along with the skin, aorta and liver tissues from transgenic animals had considerably less fat than wild type tissues. The visceral fat pads were considerably reduced or nonexistent in most transgenic animals.

In addition to the differences in epidermal morphology, the internal organs of transgenic animals were strikingly larger. Spleen and liver of homozygous transgenic mice (n=11) were the most enlarged organs; 6.3-fold for spleen and 1.9 for liver, resulting in hepatosplenomegaly developed as a consequence of PAI-1.stab expression. Hepatosplenomegaly was visually noticeable in the live transgenic animals by their puffy abdomens and dark blue spleens seen through the hairless skin. Spleen and liver were followed by enlargement seen in the heart (1.59-fold), lung (1.59-fold), and the kidney (1.24 fold).

The structural architecture of spleen was drastically changed in transgenic mice where red pulp was taken over by white pulp containing bone marrow elements such as megakaryocytes, erythroid precursors, nucleated red cells, myeloblast, and lymphoids, all of which indicated extramedullary hematopoiesis or a type of lymphoproliferative disease in the spleen. Transgenic spleen tissue was also found to contain fibroid deposits revealed by trichrome stain. Focal and sparse amyloid deposits in the Congo Red stained sections were evident under the polarized light.

Analysis of liver tissues showed that while there was no evidence of extramedullary hematopoiesis, the sinusoids were deposited with a proteinaceous material, which appeared to surround the hepatocytes. When stained with Congo Red, this proteinaceous deposit in the liver tissue sections yielded an apple green birefringence under polarized light that is typical of amyloid deposits. Kidneys from transgenic animals appeared to have enlarged glomeruli that had more fibrosis relative to wild type mice. Some of the glomeruli in the kidneys from the transgenic mice also had amyloids deposited as revealed by the Congo Red stained sections. Histochemical analysis of heart, aorta, and brain tissues did not show any remarkable differences as compared to wild type mice.

When bone marrow from transgenic animals was compared to that of wild type, no striking differences in cellularity, cell size, or shape were observed. Interestingly, no fibrosis was present in either hemizygous or homozygous transgenics (n=6). Cellular morphology of bone marrow from transgenic animals did not display any striking differences and looked as heterogenously populated as that of wild type bone marrow. No statistically significant changes in the systolic or diastolic blood pressure of transgenic mice were observed. It was observed, however, that transgenic animals older than 6 months developed spontaneous coronary arterial thrombosis and subacute myocardial infarction. In addition, lactate dehydrogenase (LDH) enzyme levels in homozygous transgenic mice (n=7) were 40% of, and in hemizygous transgenics (n=5) were 60% of, the LDH levels detected in the wild type mice (n=4).

### Example 3

### Tissue Distribution and Cellular Localization of PAI-1.stab Expression

Semi-quantitative analysis of total RNA from various organs of transgenic mice by RT-PCR revealed that the stable PAI-1 transgene was transcribed in heart, aorta, lung, and brain at considerably higher levels than the endogenous gene in wild type mice, with residual amounts of transcript detected in kidney and liver tissues. Spleen had no detectable stable PAI-1 transcript. PAI-1 ELISA and RT-PCR analyses showed that the pattern of tissue distribution of PAI-1.stab antigen followed that of PAI-1.stab mRNA. Protein extracts from dorsal skin samples had the highest levels of PAI-1 antigen (380 ng/ml) and activity (254 ng/ml), followed by comparable levels of both antigen (34 ng/ml) and activity (22 ng/ml) detected in the heart tissue. Although it was difficult to assay PAI-1 activity, protein extracts from the following tissues had detectable levels of PAI-1.stab antigen: pancreas (13 ng/ml), brain tissues (14 ng/ml), lung (40 ng/ml), kidney (12 ng/ml), liver (10 ng/ml), and spleen (5 ng/ml). Activity assays done on plasma suffered from interference when there was no substrate (t-PA) added. Upon correction for this interference, PAI-1 activity in plasma from homozygous transgenic animals was found to be 45 ng/ml, which is in agreement with the observed antigen values.

Immunohistochemical staining of dorsal skin sections localized the expression of human PAI-1 to infundibulum and inner root sheath and outer root sheath cells in the hair follicles of epidermis. Lung tissue was diffusedly stained by anti-PAI-1 antibody around the alveoli, whereas tracheal epithelia, peribronchial epithelium, and especially tracheal columnar epithelium were found to be distinctively positive for PAI-1 antigen. Endothelial cells of microvessels, valve leaflets, and aortic sinus of the heart tissue from transgenic mice were also detected by PAI-1 immunostain. In kidney tissue, endothelial cells of tubules and microvessels, and in liver, endothelial cells of capillary walls, were stained by PAI-1 antibody. Even though trichrome staining did not show any sign of fibrosis, a heterogeneous population of bone marrow cells appeared to express PAI-1.stab protein abundantly when compared to wild type bone marrow.

### Discussion of Examples 1-3

Overexpression of a stable form of human PAI-1 produced a pronounced cutaneous phenotype, as well as hepatosplenomegaly, extramedullary hematopoiesis in spleen, and systemic amyloidosis in mice. These phenotypes were observed in both lines of PAI-1.stab transgenic mice, and severity was strongly correlated with the copy number of the transgene and PAI-1 levels in the plasma. Thus, the observed phenotypes appear to be independent of the transgene integration sites and appear to be a consequence of PAI-1.stab transgene expression.

In order to target PAI-1.stab expression to vascular endothelial cells, the mPPET-1 (5.9 kb) was chosen because it has been reported to yield high levels of luciferase expression and highly specific, though not entirely limited, expression in endothelial cells of aortic tissue (Harats et al., 95 Journal of Clinical Investigation 1335-1344, 1995) in vitro and in vivo. The pattern of PAI-1.stab expression in tissues followed that of the luciferase expression in transgenic mice under the control of mPPET-1 promoter despite some differences in relative levels in each organ. The most striking difference was the level of expression that was observed in the skin as the highest versus low levels of luciferase expression observed by Harats et al. in the skin (Harats et al., 1995). This difference might be due to different strains of mice and different in vivo stabilities of luciferase and PAI-1.stab proteins.

The transgenic animals had a thickened epidermal layer compared to wild type littermates and displayed impaired keratin and pigment organization in the hair strands, thus suggesting that the regulation of keratinocyte growth and differentiation was impaired due to high levels of PAI-1.stab expressed in infundibulum and inner root sheet cells of hair follicles. Thus, the perturbation of extracellular proteolytic balance in epidermal tissue appeared to have detrimental effects on self-renewal of epidermis.

The present stable PAI-1 transgenics did not have any swollen limbs or truncated tail. Transgenic animals younger than 6 month old did not display any venous or arterial thrombosis as a result of chronicle exposure to this conformationally stable form of human PAI-1. When homozygous transgenic animals older than 6 month were characterized, it was observed that these animals had developed spontaneous coronary arterial thrombosis in the absence of hyperlipidemia, insulin resistance, or hypertension. Systemic amyloidosis was also observed over time in the PAI-1.stab transgenic mice.

Taken together, these findings indicate that PAI-1 influences a broad spectrum of processes involving cellular migration and matrix proteolysis that can impact the pathogenesis and treatment of disparate human disorders such as vascular thrombic disorders, asthma, chronic obstructive pulmonary disease, Alzheimer's Disease, myelofibrosis, wasting disorders characterized by weight loss (e.g. anorexia, AIDS, etc.), systemic amyloidosis, alopecia, male pattern baldness, glomerulosclerosis, keloids, apocrine cysts, acne, atherosclerosis, aging, a wound, and combinations thereof.

### Example 4

### Additional Transgenic Lines

This Example examined whether altering specific functional domains in human PAI-1 would prevent or reduce the extent of coronary arterial thrombosis and other complex phenotypic abnormalities. Two newly engineered lines of transgenic mice were generated employing techniques similar to those employed in Examples 1-3: one expressing human PAI-1 with impaired RCL (RCL-mutant), and another expressing human PAI-1 with impaired VN binding site (VNBS-mutant). Four (4) founder lines for both RCL- and VNBS-mutant transgenics were identified. Visual inspection of these founders showed that while VNBS-mutant mice display alopecia, RCL-mutant mice have normal hair growth. Hemizygous RCL-mutant and VNBS-mutant transgenic mice had plasma PAI-1 levels of 7.8 and 11.9 ng/ml, respectively. Although VNBS-mutants displayed hepatosplenomegaly and extramedullary hematopoiesis to the same extent as mice transgenic for PAI-1 with both functional domains, these phenotypes were negligible in the RCL-mutants.

In conclusion, the RCL or the PA inhibitory domain of PAI-1 appears to be critical in yielding the complex phenotypes observed in PAI-1 transgenic mice. These novel findings further support that PAI-1 inhibitors can be employed in the treatment of a broad spectrum of human conditions and disorders, including myelofibrosis, amyloidosis, and hair loss.

### Example 5

### VOD in Mice Treated with a Nitric Oxide Synthase Inhibitor

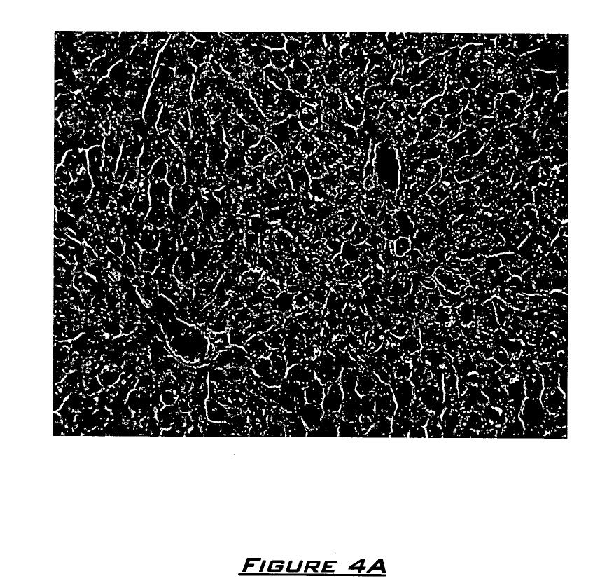

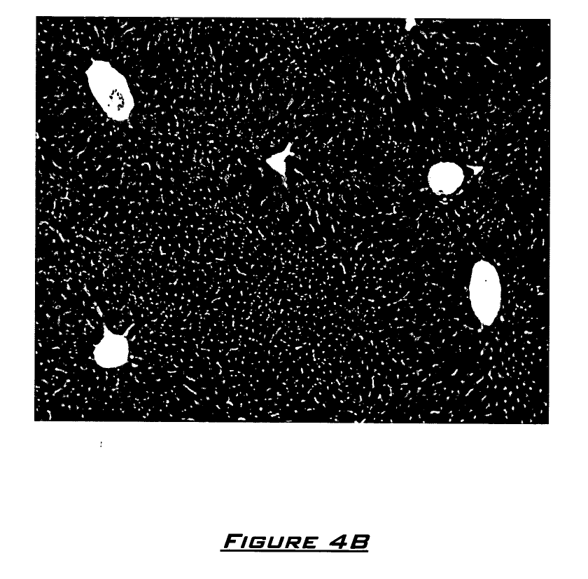

Chronic inhibition of nitric oxide synthase (NOS) was achieved using an oral inhibitor of NOS, N(Ω)-nitro-L-arginine methyl ester (L-NAME; Sigma-Aldrich Co., St. Louis, Mo., United States of America), given in drinking water at 1.0 mg/L for up to six weeks. Upon sacrifice, the tissues were immediately fixed in formalin or frozen in liquid nitrogen. Formalin fixed tissues were processed and embedded in paraffin, sectioned at 5 μm, and stained with Masson's trichrome stain to visualize fibrin cloth and overall tissue morphology. As shown in FIGS. 4A and 4B, veno-occlusive thrombi were observed in wild type mice that received L-NAME while PAI-1 deficient mice (Carmaliet et al., 1993) appeared to be protected from this effect.

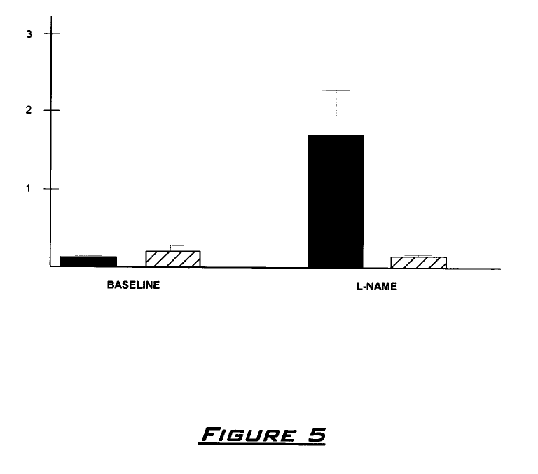

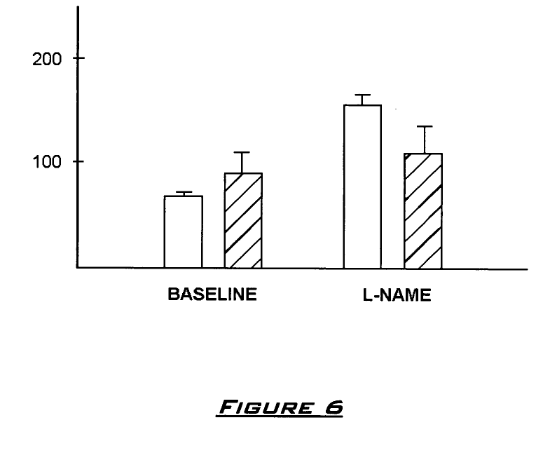

To evaluate hepatic function under chronic inhibition of NOS, plasma levels of total bilirubin and aspartate amino transferase (AST) were determined in wild type and in PAI-1 deficient mice. The results of these experiments are presented in FIGS. 5 and 6.

### Example 6

### VOD in Mice Treated with a Nitric Oxide Synthase Inhibitor and a PAI-1 Antagonist, Tiplaxtinin (PAI-039)

Animals. PAI-1^{-/-} mice (Carmaliet et al., 1993), and wild type mice on the same genetic background (C57BL/6J) were purchased from the Jackson Laboratory (Bar Harbor, Me., United States of America). Six male animals were studied in each of three experimental groups. L-NAME (Sigma-Aldrich Co.) is a nonselective reversible inhibitor of nitric oxide synthase (Hobbs et al., 1999), and was administered as described in Example 5 (see also Kaikita et al., 2001). Animals were fed a regular chow diet, except those wild type animals that received tiplaxtinin (PAI-039; Elokdah et al., 2004) mixed into the chow (1.0 mg/g) in addition to L-NAME. This dose has been previously shown to produce steady-state plasma levels of tiplaxtinin nearly equivalent to the in vitro IC₅₀ against PAI-1 (Weisberg et al., 2005). Systolic blood pressure was serially determined as described in Kaikita et al., 2001.

Histopathology. Six weeks after the initiation of L-NAME treatment, animals were euthanized for gross and microscopic hepatic analyses. After extensive saline perfusion, livers were harvested, formalin fixed, and embedded in paraffin blocks. Hepatic sections were stained with Masson's trichrome, and hematoxylin and eosin stains, and photographed under 20×-80× magnification using an Olympus BX40 microscope with an Optronics Magnafire digital camera (Meyer Instrucments, Houston, exas, United States of America). Digital image analysis of each photomicrograph was performed with IMAGEPRO® PLUS software (MediaCybernetics, Silver Spring, Md., United States of America). The extent of hepatic venous thrombosis was determined by calculating the vascular luminal area obstructed by thrombi divided by the total vascular area in any given 20× field. For each liver, the obstructed and total vascular areas were calculated from five random 20× fields. In total, 240 individual veins were analyzed in each of the treatment groups. Sections were examined and characterized by a single blinded investigator.

Clinical Chemistry. Blood samples were taken by retro-orbital bleeding at week 0 and upon euthanasia. Samples were anti-coagulated using acidified 3.8% sodium citrate. AST and bilirubin tests were performed at the Vanderbilt Clinical Diagnostics Laboratory (Vanderbilt University, Nashville, Tenn., United States of America) per clinical protocols. Plasma PAI-1 activity was measured using a functional ELISA assay that identifies only the active protein (described in Ngo et al., 1999, and available from Molecular Innovations, Inc., Southfield, Mich., United States of America).

Statistical Analysis. Data was analyzed by analysis of variance (ANOVA) and was performed by using SPSS® 11.0 (SPSS Inc. Chicago, Ill., United States of America). When ANOVA indicated a statistically significant difference between treatment groups, Scheffe's multiple comparison procedure was then used to determine which pairs of treatment groups were significantly different. Data are reported as the mean±standard error of the mean (SEM).

### Example 7

### NOS Inhibition Induces Hepatic Venous Thrombosis in Wild Type Mice

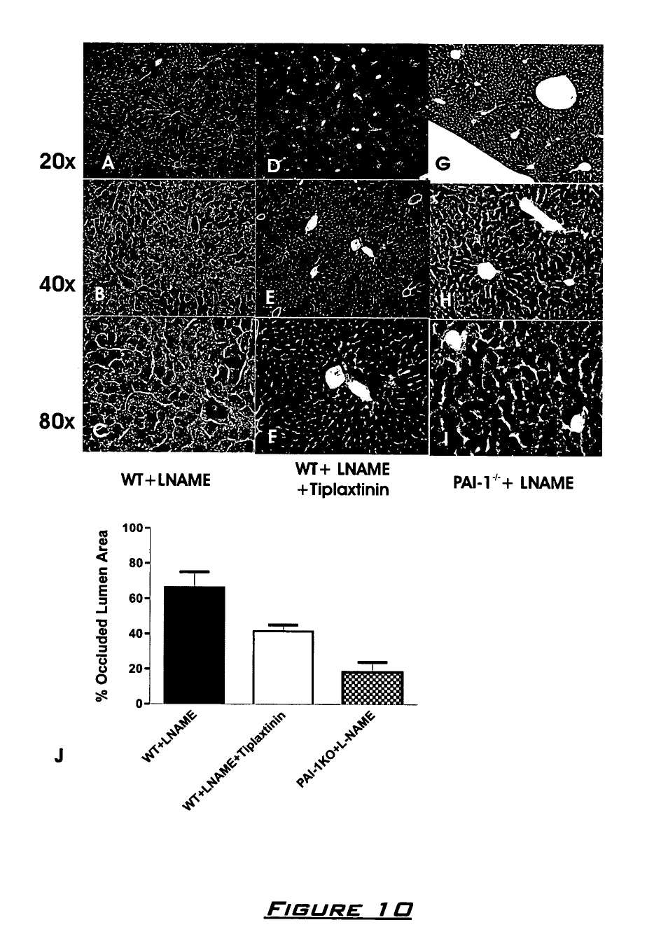

At baseline, there were no significant differences in systolic blood pressure between groups. After 6 weeks, systolic blood pressure was significantly higher in L-NAME-treated wild type mice, compared to L-NAME-treated PAI-1^{-/-} mice (140.7±5.0 mm Hg in wild type mice vs. 121.4±7.3 mm Hg in PAI-1^{-/-} mice; p<0.001). At the time of euthanasia, livers from L-NAME-treated wild type mice exhibited a significant number of hepatic and portal veins occluded by fibrin thrombi compared to the L-NAME-treated PAI-1^{-/-} mice (66.43±8.7% occluded area in wild type mice vs. 18.36±5.6% occluded area in PAI-1^{-/-} mice; p<0.001). Wild type mice receiving L-NAME also exhibited other histologic changes associated with VOD, including hepatocyte necrosis and fibrosis. In contrast, these changes were not apparent in PAI-1^{-/-} mice receiving L-NAME (FIGS. 10G-I).

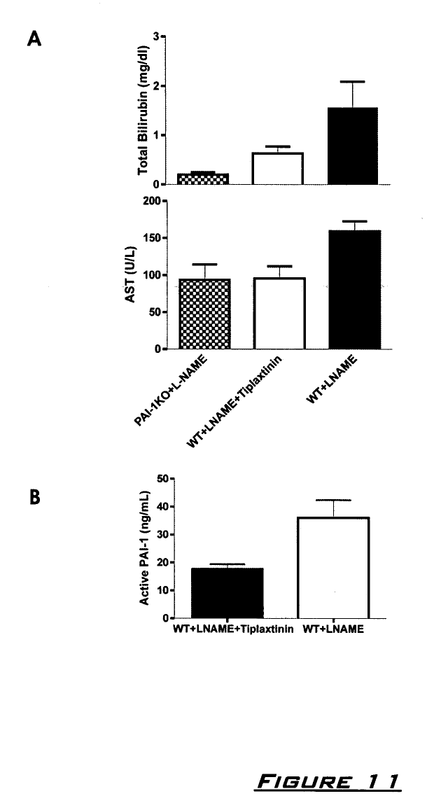

There were no significant differences in AST or bilirubin between the treatment groups at baseline. After 6 weeks of L-NAME treatment, both AST (159.4±13.5 U/L in wild type mice vs. 93.8±20.5 U/L in PAI-1^{-/-} mice; p=0.018) and bilirubin (1.56±0.5 mg/dl in wild type mice vs. 0.20±0.05 mg/dl in PAI-1^{-/-} mice; p<0.001) were increased in wild type mice compared to PAI-1^{-/-} mice (FIG. 11A). Elevated levels of AST and bilirubin are associated with VOD in humans, and reflect injury to hepatocytes and obstruction of the liver. In this model, wild type mice exhibited similar increases in serum total bilirubin and AST, while PAI-1^{-/-} mice do not, suggesting that PAI-1^{-/-} deficiency is sufficient to protect against hepatic injury despite decreased nitric oxide (NO). Furthermore, the presently disclosed data suggested that PAI-1 plays an early role in the pathogenesis of VOD and might provide insight into the sequence by which endothelial damage leads to hepatic thrombosis. It is likely that increases in serum total bilirubin reflected the formation of obstructive hepatic and portal venous thrombi that resulted from damage to endothelial cells, while changes in AST occurred subsequently and reflected the resulting hepatocyte injury.

### Example 8

### Effects of PAI-1 Inhibition by Tiplaxtinin (PAI-039) in L-NAME-Treated Mice

As shown in FIG. 10, L-NAME induced numerous and extensive hepatic thrombi in wild type mice. Consistent with the data observed in PAI-1^{-/-} mice, tiplaxtinin, a small molecule inhibitor of PAI-1, significantly attenuated the number and extent of L-NAME induced venous thrombi in wild type mice (41.55±3.6% occluded area in wild type mice vs. 66.43±8.7% occluded area in PAI-1^{-/-} mice; p<0.05; see FIG. 10J).

Plasma PAI-1 activity was decreased in wild type mice receiving tiplaxtinin and L-NAME compared to those mice that received L-NAME alone (17.68±1.6 ng/ml in wild type mice vs. 36.05±6.34 ng/ml in PAI-1^{-/-} mice; p=0.011; see FIG. 11B), and had no effect on L-NAME induced increases in systolic blood pressure (136.6±11.7 mm Hg in wild type mice vs. 140.8±11.74 mm Hg in PAI-1^{-/-} mice, p>0.05). The consistency of the findings that both pharmacological inhibition and genetic deletion of PAI-1 reduce the extent and severity of hepatic venous thrombi suggested that PAI-1 is directly involved in the molecular pathogenesis the disease.

### Example 9

### Non-Alcoholic Steatohepatitis (NASH) in Mice Fed a Fat-Enriched Diet

Wild type and PAI-1 deficient mice were fed a diet enriched in fat. In addition to dramatic weight gain, mice in both groups showed evidence of fatty infiltration of the liver. While mice in the wild type group showed evidence of the development of progressive hepatic fibrosis, PAI-1 deficient mice in general were protected from the development of hepatic fibrosis and injury.

### Example 10

### PAI-1 Involvement in Bone Synthesis

Micro-computed tomography (mCT) was used to determine morphological and microarchitectural features of skeletal system in live mice under anesthesia and with no contrasting agent. For each mouse, mCT X-ray projections were acquired with a 25 μm resolution to scan the whole body. The X-ray source was rotated incrementally around the mouse scanned to generate a two-dimensional image of each slice. These contiguous slices were stacked in an order to form the three-dimensional image of the whole mouse skeleton. The X-ray attenuation coefficients (a function of the bone mineral density) of femur bone from age- and sex-matched wild type and transgenic mice were measured. The cortical thickness of femur bone from wild type and transgenic animals were also measured using sagittal X-ray images.

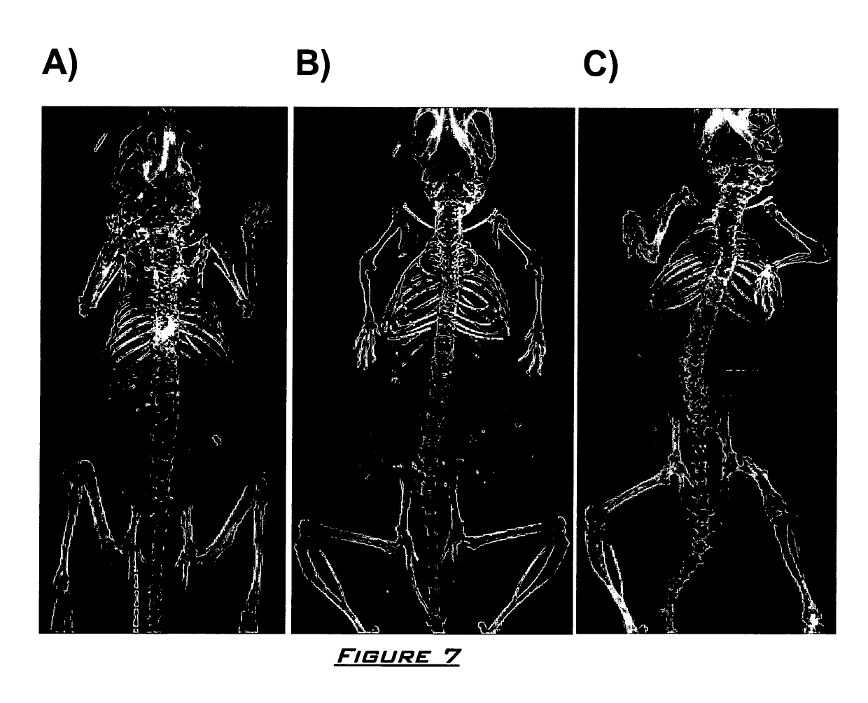

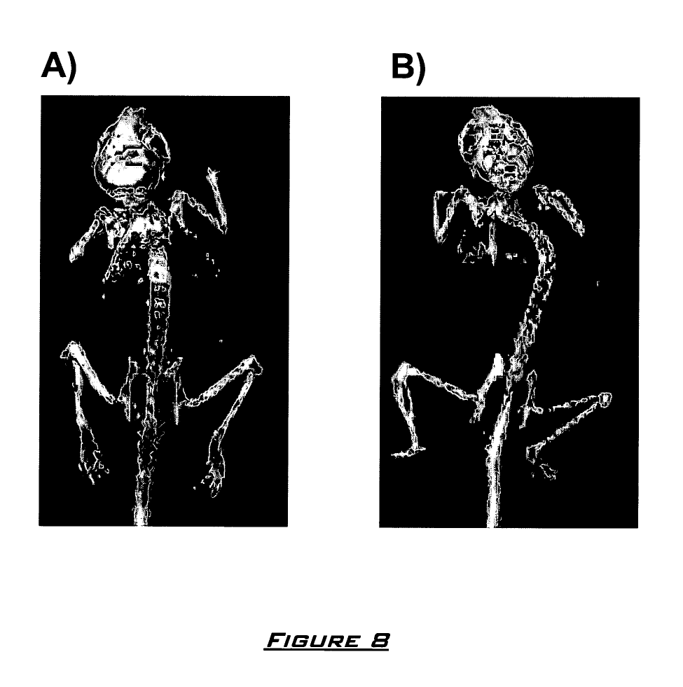

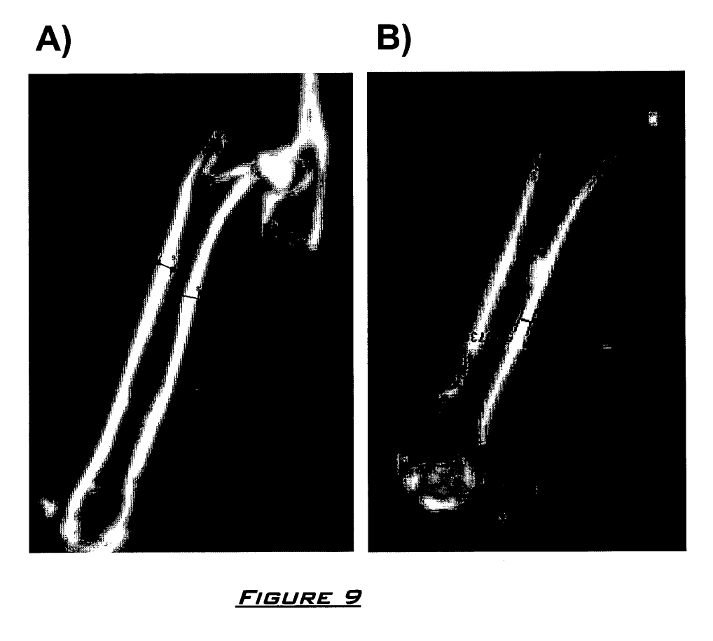

As shown in FIGS. 7A-9B, evidence was found by mCT of extensive osteoporosis and osteopenia in PAI-1 transgenic animals. This was associated with a nearly 50% reduction in bone mass, a 32% reduction in thickness of the femoral cortex, and by widespread evidence of general bony erosion.

### Example 11

### Polycystic Ovarian Syndrome (PCOS) in PAI-1 Transgenic Mice

Female transgenic mice that overexpressed a stable form of human PAI-1 showed dramatic evidence of ovarian enlargement and the presence of large cystic structures.

### Discussion of Examples 5-11

Veno-occlusive disease and thrombosis. Thrombosis of the hepatic and portal veins occurs rather frequently in patients that have undergone bone marrow transplantation. Up to 50% of patients that undergo bone marrow transplantations suffer some degree of veno-occlusive disease (VOD), and this disorder has a mortality rate as high as 49% in some series. It is thought to be caused by high doses of chemotherapy and radiation.

Experimental treatments for VOD include the administration of thrombolytic agents and low-molecular weight heparin. Interestingly, elevated levels of plasminogen activator inhibitor (PAI-1) have been reported to aid in the diagnosis of patients with VOD. In the experiments discussed in Example 5, an oral inhibitor of nitric oxide synthase (L-NAME) was chronically administered to mice. Hepatic venous thrombosis was found to occur quite frequently in wild type mice given an oral inhibitor of L-NAME in their drinking water for up to six weeks. In contrast, PAI-1 deficient animals showed little or no evidence of hepatic vein thrombosis when treated with L-NAME. Taken together, these data suggest that PAI-1 antagonists can be of therapeutic value in the prevention and treatment of VOD in humans.

In summary, the presently disclosed subject matter provides direct evidence that PAI-1 is more than a biochemical marker of VOD. Indeed, the results presented herein establish that PAI-1 plays a role the pathogenesis of hepatic venous occlusive disease. Since both genetic deficiency and pharmacological inhibition of PAI-1 provided protection against hepatic thrombosis, the presently disclosed subject matter also provides for the development of pharmacological antagonists of PAI-1 for the treatment of VOD. Importantly, while other chemical classes of PAI-1 inhibitors have been reported that include both direct-acting small-molecule inhibitors and antibodies (see e.g., Heymans et al., 1999; Elokdah et al.), none has shown the oral activity and efficacy of tiplaxtinin or has been profiled in a model of this disease. The presently disclosed subject matter also indicates that PAI-1 is a rational and druggable target for the prevention and treatment of VOD in humans.

Obesity and fatty infiltration of the liver. The epidemic of obesity has brought with it the emergence of a number of different clinical disorders, including the development of fatty infiltration of the liver. In some individuals, this is associated with progressive liver fibrosis, hepatic failure, and even hepatocellular carcinoma. In fact, at the current time, non-alcoholic fatty liver disease (also referred to as non-alcoholic steatohepatitis (NASH)) brought on by obesity is the second leading cause of hepatic failure in this country. In the experiments discussed in Example 9, wild type and PAI-1 deficient mice were fed a diet enriched in fat. Associated with dramatic weight gain, mice in both groups showed evidence of fatty infiltration of the liver. While mice in the wild type group showed evidence of the development of progressive hepatic fibrosis, PAI-1 deficient mice in general were protected from the development of hepatic fibrosis and injury.

Osteoporosis and osteopenia. Osteoporosis is a common disorder that afflicts many postmenopausal women and is a common cause of morbidity and mortality in the aged population. Current therapies to prevent and treat osteoporosis include hormonal therapies and calcium supplementation. In the experiments discussed in Example 10, evidence was found by mCT of extensive osteoporosis and osteopenia in PAI-1 transgenic animals. This was associated with a nearly 50% reduction in bone mass, a 32% reduction in thickness of the femoral cortex, and by widespread evidence of general bony erosion. These findings suggest that PAI-1 overproduction contributes to the development of osteoporosis in mammals, and suggests that PAI-1 antagonists can be of value in the prevention and treatment of osteoporosis in humans.

Polycystic ovarian syndrome (PCOS). Polycystic ovarian syndrome (PCOS) is a relatively common disorder associated with insulin resistance and elevated levels of plasma PAI-1. In the experiments discussed in Example 11, female mice that overexpressed a stable form of human PAI-1 showed dramatic evidence of ovarian enlargement and the presence of large cystic structures. This suggests that the elevation in PAI-1 seen in PCOS is not coincidental, but rather contributes to the pathogenesis of this disorder in mammals. Based on these findings, PAI-1 antagonists can be of value in the prevention and/or treatment of PCOS in humans.

### REFERENCES

The references listed below (which are also cited hereinabove) as well as all references cited in the specification are incorporated herein by reference to the extent that they supplement, explain, provide a background for, or teach methodology, techniques, and/or compositions employed herein.
Aertgeerts et al., 113 Journal of Structural Biology 239-245, 1994.
Alessi et al., 60 Thrombosis & Haemostasis 60:491-494, 1988.
Auwerx et al., 8 Arteriosclerosis 68-72, 1988.
Barany, 37 Gene 111-123,1985.
Barton et al., 18 Nucleic Acids Research 7349-7355, 1990.
Berkenpas et al., 14 EMBO J. 2969-2977, 1995.
Blackwell et al., 1 Molecular Medicine 194-205, 1995.
Booth, in Bloom et al. (eds.) Haemostasis and Thrombosis, 3^{rd} ed, Churchill Livingstone, Edinburgh, Scotland, United Kingdom, 1994, pp. 699-717.
Carmeliet et al., Journal of Clinical Investigation 2746-2755, 1993.
Capecchi, 244 Science 1288-1292, 1989.
Chomiki et al., 72 Thrombosis & Haemostasis 44-53, 1994.
Colicelli et al., 199 Molecular and General Genetics 537-539, 1985.
Colucci et al., 75 Journal of Clinical Investigation 818-824, 1985.
Dawson et al., 11 Arteriosclerosis & Thrombosis 183-190, 1991.
Dawson et al., 268 Journal of Biological Chemistry 10739-10745, 1993.
Declerck et al., 263 Journal of Biological Chemistry 15454-15461, 1988a.
Declerck et al., 71 Blood 220-225, 1988b.
Dichek and Quertermous, 74 Blood 222-228, 1989.
Ding et al., 272 Journal of Biological Chemistry 28142-28148, 1997.
Elokdah et al., 47 Journal of Medicinal Chemistry 3491-3494, 2004.
Emeis and Kooistra, 163 Journal of Experimental Medicine 1260-1266, 1986.
Feener et al., 95 Journal of Clinical Investigation 1353-1362, 1995.
Francis et al., 115 American Heart Journal 776-780, 1988.
Gabazza et al., 177 Lung 253-262, 1999.
Gustin et al., 14 Biotechniques 22, 1993.
Harats et al., 95 Journal of Clinical Investigation 1335-1344, 1995.
Hermans et al., 354 Lancet 556-560, 1999.
Heymans et al., 5 Nature Medicine 1135-1142, 1999.
Hobbs et al., 39 Annual Review of Pharmacology and Toxicology 191-220, 1999.
Jones and Winistorfer, 12 Biotechniques 528-530, 1992.
Joulin and Richard-Foy, 232 European Journal of Biochemistry 620-626, 1995.
Juhan-Vague et al., 57 Thrombosis & Haemostasis 67-72, 1987.
Kaikita et al., 104 Circulation 839-844, 2001.
Keijer et al., 266 Journal of Biological Chemistry 10700-10707, 1991.
Keown et al., 185 Methods in Enzymology 527-537, 1990.
Kruithof, 40 Enzyme 113-121, 1988.
Kruithof et al., 64 Blood 907-913, 1984.
Li and Cohen, 85 Cell 319-329, 1996.
Loskutoff et al., 32 Journal of Cellular Biochemistry 273-280, 1986.
Luyckx et al., 96 Proceedings of the National Academy of Sciences of the United States of America 12174-12179, 1999.
Mansfield et al., 71 Thrombosis & Haemostasis 731-736, 1994.
Margaglione et al., 14 Arteriosclerosis & Thrombosis 1741-1745, 1994.
Marotti and Tomich, 6 Gene Analysis Techniques 67-70, 1989.
Mortlock et al., 6 Genome Research 327-333, 1996.
Nar et al., 297 Journal of Molecular Biology 683-95, 2000.
Ngo and Declerck 82 Thrombosis & Haemostasis 1510-1515, 1999.
Ngo et al., 79 Thrombosis & Haemostasis 808-812, 1998.
PCT International Publication No. WO 93/25521.
PCT International Publication No. WO 01/51085.
Prentid et al., 29 Gene 303-313, 1984.
Rosenberg and Aird, 340 New England Journal of Medicine 1555-1564, 1999.
Samad et al., 97 Journal of Clinical Investigation 37-46, 1996.
Sambrook and Russell, Molecular Cloning: A Laboratory Manual (Third Edition), Cold Spring Harbor Laboratory Press, Cold Spring Harbor, N.Y., United States of America, 2001.
Sauer, 14 Methods 381-392, 1998.
Sawdey et al., 264 Journal of Biological Chemistry 10396-10401, 1989.
Sayers et al., 13 Biotechniques 592-596, 1992.
Sprengers and Kluft, 69 Blood 381-387, 1987.
Stefansson and Lawrence, 383 Nature 441-443, 1996.
Thogersen et al., 98 Circulation 2241-2247, 1988.
Thomas and Capecchi, 346 Nature 847-850, 1990.
U.S. Pat. No. 4,736,866
U.S. Pat. No. 5,162,215
U.S. Pat. No. 5,234,933
U.S. Pat. No. 5,326,902
U.S. Pat. No. 5,422,245
U.S. Pat. No. 5,489,742
U.S. Pat. No. 5,550,316
U.S. Pat. No. 5,573,933
U.S. Pat. No. 5,614,396
U.S. Pat. No. 5,625,125
U.S. Pat. No. 5,639,726
U.S. Pat. No. 5,648,061
U.S. Pat. No. 5,741,957
U.S. Pat. No. 5,980,938
U.S. Pat. No. 6,248,743
Vaughan et al., 67 Circulation Research 1281-1286, 1990.
Vaughan et al., 95 Journal of Clinical Investigation 995-1001, 1995.
Vaughan et al., 96 Circulation 442-447, 1997.
Weiner et al., 126 Gene 35-41, 1993.
Weisberg et al., 25 Arteriosclerosis, Thrombosis, and Vascular Biology 365-371, 2005.
Wiman et al., 105 Journal of Laboratory and Clinical Medicine 265-270, 1985.
Wyss-Coray et al., 147 American Journal of Pathology 53-67, 1995.
Zhu, 177 Analytical Biochemistry 120-124, 1989.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method of treating a warm-blooded vertebrate animal having a medical condition associated with PAI-1 biological activity, said method comprising:
(a) administering a treatment effective amount of a composition that exhibits PAI-1 inhibition activity to a warm-blooded animal having a medical condition associated with PAI-1 biological activity; and
(b) observing an improvement in the medical condition indicative of inhibition activity of PAI-1 in the warm-blooded animal having the medical condition.

2. The method of claim 1, wherein the warm-blooded vertebrate animal having the medical condition is a mammal or a bird.

3. The method of claim 2, wherein the mammal is selected from the group consisting of a rodent, a swine, a ruminant, and a primate.

4. The method of claim 3, wherein the primate is a human.

5. The method of claim 1, wherein the medical condition associated with PAI-1 biological activity is selected from the group consisting of veno-occlusive disease (VOD), pulmonary fibrosis, obesity, non-alcoholic steatohepatitis (NASH), osteoporosis, osteopenia, polycystic ovarian syndrome (PCOS), and combinations thereof.

6. The method of claim 5, wherein the medical condition associated with PAI-1 biological activity is veno-occlusive disease (VOD).

7. The method of claim 5, wherein the medical condition associated with PAI-1 biological activity is selected from the group consisting of obesity, non-alcoholic steatohepatitis (NASH), and combinations thereof.

8. The method of claim 5, wherein the medical condition associated with PAI-1 biological activity is selected from the group consisting of osteoporosis, osteopenia, and combinations thereof.

9. The method of claim 1, further comprising obtaining a composition that exhibits PAI-1 inhibition activity in a transgenic non-human warm-blooded vertebrate animal having incorporated into its genome a PAI-1 gene, the PAI-1 gene being present in said genome in a copy number effective to confer overexpression of a PAI-1 polypeptide in the transgenic non-human animal.

10. A method of hepatoprotection, the method comprising:
(a) administering a treatment effective amount of a composition that exhibits PAI-1 inhibition activity to a warm-blooded vertebrate animal in need of hepatoprotection and having a medical condition associated with PAI-1 biological activity; and
(b) observing an improvement in the medical condition indicative of inhibition activity of PAI-1 in the warm-blooded animal having the medical condition.

11. The method of claim 10, wherein the warm blooded vertebrate animal is a mammal or a bird.

12. The method of claim 11, wherein the mammal is selected from the group consisting of a rodent, a swine, a ruminant, and a primate.

13. The method of claim 12, wherein the primate is a human.

14. The method of claim 10, wherein the medical condition associated with PAI-1 biological activity is selected from the group consisting of veno-occlusive disease (VOD), pulmonary fibrosis, obesity, non-alcoholic steatohepatitis (NASH), and combinations thereof.

15. The method of claim 14, wherein the medical condition associated with PAI-1 biological activity is veno-occlusive disease (VOD).

16. The method of claim 14, wherein the medical condition associated with PAI-1 biological activity is selected from the group consisting of obesity, non-alcoholic steatohepatitis (NASH), and combinations thereof.

17. The method of claim 10, further comprising obtaining a composition that exhibits PAI-1 inhibition activity in a transgenic non-human warm-blooded vertebrate animal having incorporated into its genome a PAI-1 gene, the PAI-1 gene being present in said genome in a copy number effective to confer overexpression of a PAI-1 polypeptide in the transgenic non-human animal.

18. A method of treating a warm-blooded vertebrate animal having veno-occlusive disease (VOD) associated with PAI-1 biological activity, said method comprising:
(a) administering a treatment effective amount of a composition that exhibits PAI-1 inhibition activity to a warm-blooded animal having veno-occlusive disease (VOD) associated with PAI-1 biological activity; and
(b) observing an improvement in the VOD indicative of inhibition activity of PAI-1 in the warm-blooded animal.

19. The method of claim 18, wherein the warm-blooded vertebrate animal is a human.

20. The method of claim 18, wherein the composition that exhibits PAI-1 inhibition activity comprises tiplaxtinin (PAI-039; 2-{1-Benzyl-5-[4-(trifluoromethoxy)phenyl]-1H-indol-3-yl}-2-oxoacetic acid) or a pharmaceutically acceptable salt thereof.


SEQUENCE LISTING (16 sequences)

SEQ ID NO: 1 - DNA, 2970 bp, Cow
  CDS (122)..(1330)
gcaaagagcaccgtccagagagagccacacctccttttcagtggaggaagggctaccaccgtcaacacccgcagcggcagcaggaacaaaccgcccatcccaaggcactgcgaaattcaggatgcggatgtctccggtctttgcctgcctcgccctgggcctggccctcatctttggtgaagggtctgcctcctaccagccccagtctgcggcggccagcctggccacagactttggagtgaaggtgtttcagcaggtggtgcgggcctccaaggaccgcaacgtggttttctcaccctatggggtggcctcagtcctggccatgctgcagctgaccacaggaggagaaacccgccagcagatccaagaggcaatgcaattcaagattgaagagaagggcatggcccctgccttccaccgactgtacaaggagctcatgggcccgtggaacaaggatgagatcagcacagccgatgccatcttcgtgcagcgggacctagagctggtccatggtttcatgcccaacttcttcaggctgttccgtaccacggtcaagcaggttgacttctctgaagtggagagagccaggttcatcgtcaacgactgggtgaaaagacacacaaaaggcatgatcagcgacttacttggtgaaggggctgtggaccagctgacacgcctggtcctggtaaatgccctctacttcaacggccagtggaagatgcccttcccagagtcaaacacccaccaccgcctcttccacaagtccgatggcagcaccatctctgtgcccatgatggctcagaccaacaagttcaactacactgagtttaccacccccgacggccgttactacgacatcctggaattgccctaccacgggaacactctcagcatgctcattgctgccccctatgagaaggaggtgccgctctctgccctcaccagcattctggatgctgagctcatcagccagtggaaagggaatatgaccaggctgacccgcctcctggttctgcccaagttctccctggagaccgaaatcgacctcaggaggccgctggagaatttgggaatgaccgacatgtttaggccgagccaggcggacttctccagtttttcagatcaagagtttctgtacgtgtcgcaggcgctgcagaaggtgaagattgaggtgaatgagagcggcacgctggcgtcctcctccacagcccttgtagtctcagcccgaatggcccccgaggagatcatcatggacagacccttcctcttcgtggtgcggcacaatcccacaggaactgtcctgttcatgggccaagtgatggaaccctgaccatggggaaggcagccctcatctgggacagaatggagatgtccaagaggaagaaagtccggagcaaagaatttttattaattcatttttctggaaaaagagaagatgtttatttatttatttttccatggtaaattcttttgaatctgcctcttagacctaactttgggctctctcaggaggggcaaagaggacctttgagttaaaccctccaatggagaccctgggaaagactgggaggcataacacccagccggcctcccaactggactgtaggactcccaggactgctggcccagctgcttctgcccatcgttctgcctggttcggttttgggtcctggatcccaccgaggccctggtaggatggcaccacaaggcctacatgaaggagcttttgtgtgttcactagaaatgtgtgttccggtcacgttgctgtcattcttgcactgtctgccactgctgagaaggctggcagcaggcccgagaaggccaaggcgagaaacaccctttcatgccaaggtccatctgtccccagctccgaggtttgagacccacccaggcctggctgctccctccccaggaaacagtgtgtatatattattttagagtgtagatgacttgtttactcagagaagcaggtttctgcttcccacaaactttatgttgcagaaacgcaaggagagacaaggtgtgtgcctggttctttggctcccatctcctggtggggagggtgagatgccaggggtgtgcctgaatatttatcacatccttgtccttgtgtgcttgggagaaagaaaggtctactgagaaaacagattatttagccttgttcaccgtgttccctttggggggtctgtgtcgccgcatctcaggagaggcctcttgactgtccctcccctccaccaggtggcaagcctcccggggcccacactgccacctggcggaggcccagcgcccccgcgcctctcctttcctcgattttccacccgatggagccgcgtccctggcaggaccatccaacttcggctcacttttagggaccgaaaggatgtggtgggtgaagggagacggagtggtttcaaaattttccagtatatttaggagcgggagtgcaaggggctccacgacctagcaggacagaactttccccaattacagggtgactcacagccgcactggtgactcatttcaatgtgtcatttccggctgctgtgtgtgagcggtggacgcgtgagagagagagagagagagagaatgagagagacggcgagctcgggctcaattacctccggcagataatctttctgacagccagctagctgagggggtacagagagaggaccgatttactgaagaattgcacagagatgccgaatgaatgtaacctaatagaaccctaatcaccccgctgtgcccttcagcgaaaactctcccttctttgtgtgtatgttttgttttgtttttcttttttcttgatgcactggacagtgacagccacactcagtacccccacgtgtggggtccatggctcttgaaattgctttttcacttttgatatagaagcaagtaaaaaaaaatgttttttaaaaattaataataaataaataaaaagaatattccaaaataaaaaaaaaaaaa

SEQ ID NO: 2 - protein, 402 aa, Cow
MRMSPVFACLALGLALIFGEGSASYQPQSAAASLATDFGVKVFQQVVRASKDRNVVFSPYGVASVLAMLQLTTGGETRQQIQEAMQFKIEEKGMAPAFHRLYKELMGPWNKDEISTADAIFVQRDLELVHGFMPNFFRLFRTTVKQVDFSEVERARFIVNDWVKRHTKGMISDLLGEGAVDQLTRLVLVNALYFNGQWKMPFPESNTHHRLFHKSDGSTISVPMMAQTNKFNYTEFTTPDGRYYDILELPYHGNTLSMLIAAPYEKEVPLSALTSILDAELISQWKGNMTRLTRLLVLPKFSLETEIDLRRPLENLGMTDMFRPSQADFSSFSDQEFLYVSQALQKVKIEVNESGTLASSSTALVVSARMAPEEIIMDRPFLFVVRHNPTGTVLFMGQVMEP

SEQ ID NO: 3 - DNA, 2876 bp, Human
  CDS (76)..(1284)
gaattcctgcagctcagcagccgccgccagagcaggacgaaccgccaatcgcaaggcacctctgagaacttcaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagc

SEQ ID NO: 4 - protein, 402 aa, Human [sequence omitted]

SEQ ID NO: 5 - DNA, 1962 bp, Human
  CDS (1)..(1161)
gtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggacggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaaggtcagatgcgtgcccagctcttcaccccccaatctcttggtggggaggggtgtacctaaatatttatcatatccttgcc

SEQ ID NO: 6 - protein, 386 aa, Human
VFGEGSAVHHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISNLLGKGAVDQLTRLVLVNALYFNGQWKTPFPDSSTHRRLFHKSDGSTVSVPMMAQTNKFNYTEFTTPDGHYYDILELPYHGDTLSMFIAAPYEKEVPLSALTNILSAQLISHWKGNMTRLPRLLVLPKFSLETEVDLRKPLENLGMTDMFRQFQADFTSLSDQEPLHVAQALQKVKIEVNESGTVASSSTAVIVSARMAPEEIIMDRPFLFVVRHNPTGTVLFMGQVMEP

SEQ ID NO: 7 - DNA, 2970 bp, Mink
  CDS (69)..(1271)
tcgccaccgtcagcagcagcagcagcaggacagaccagcaatcacaaggcacctttgagaacttcaggatgcagatgtctacagtctgccttgccctgggcctggcccttgtctttggtgaagcatcggcctcctacctccacgagacccgggcagcggaactggccacagacttcggagtgaaagtgtttaagcaggtggcacaggcctccaaggaccgcaacatggttttttccccctatgggttggcctctgtcctggccatgttgcagctgaccacagcaggagagacccggcagcagatccaagaggccatgcggttccagattgatgagaagggcatggcacctgccctccgccaactgtacaaggaactcatggggccgtggaacaaggatgagatcagtaccgcggacgccatcttcgtccagcgggatttgaagctggtccacggcttcatgccctacttcttcaggctgttccaaaccacagtcaagcaggtggacttctcagaggtggagagagccaggttcatcatcaacgactgggtgaagcgacacacaaaaggcatgattggcgacctgctgggcagagggactgtggaccagctgacgcgtctgatgctggtgaatgccctctacttcaacggccagtggaagacccctttccccaagtcgggcacccaccaccgcctcttccacaaatctgatggcagcaccgtctccgtgcccatgatggctcagaccaacaagttcaactacaccgagttttctacccccgagggccgttattacgacatcctggaactgccctatcacggagacacgctcagcatgttcattgctgctccctatgaaaaagacgtgcctctttctgccctcaccaacatcctggatgcccagctcatcagccagtggaaagggaatatgaccagacggctccgcctcctggttctgcccaagttctccctggagagcgaagtcaacctccggggacccctggagaacttggggatgactgacatgttcaggccaaaccaggcagacttctccagtctttcagatcaagaggcactgtacgtgtcccaggcgctgcagaaagtgaaaatcgaggtgaacgagagcggcacggtggcgtcctcctctacagccatcatcgtctcagcccgaatggcccccgaggagatcatcatggacagacccttcctcttcgtggtgcggcacaaccccacaggaacggtccttttcatgggccaagtgatggaaccctgaccgcgagcacagcagccctcatctgggacagaactggagatacatccaagaagaaggaactctaaaggaacaaaatatatattttttcattaatttttctggagaaaaagaagtcatttgccctttagggaaaaacaaaacaaaacaaaaaccaaaaaaacaaaaaaaggtaaatctttcgaatctgcatcccagacctcagcctctcccaggaatgggaaagaggacctttcagtaaaactccacggtggagccccccggagagacctccgaagcacagctgggtctccacaccagactgcaaacctcagacaaccactggcgcagtgctctgcccatccgggtctgcagacctggaccccgccgagaccccggcaggatggcaccccaaggcttgcgggagcttttgtgtgcttggtggaaacgatttgtgttccaggcacgtagctgtcactcctgcactgtctgccactgctgaggaggctggcggcgggccaaagaaggccagtgggagaggcaccctttctgttcgaggtctgtgccgccccgatcggacggtctcgcggccccgggcggagacccgccctgacctggccgtggccctccccagaaacagtgtgcatatattattttggagtgtaggtgacttgtttactcataggagcaggtttctgcttcccactaactttattttgcaggaatggaggaatagaagtgagatgcgtgcctggttcttggctctaatctcccggtggggagggtgggatgccaggggtgtgcttgggtatttaatcacatccttgtccttgtgtgcttgtgagagagaaagaggactctcaagaaaaacgtagtctatgtatttgcttgtcatgttccctggtggtttgtgtcttgcctctcaggagtctggccccctgactagctgcccctctgggcggcaagcgtggtgggacccacactgcccccttgtggcttcctgagccccgtggttcctctcccgtcctggcccttccactccatggagaagacccccgtcaagatgatctaactttagggaccaaaaggatgtggtgggtcagagagattacagcgaggacagcatgctttcaaaattttccaatatatttaggaacaggagagcaaggggctgcacgacctaacaggacagaactttccccaattacagggtgattcacagccgcattggtgactcatttcgatgtgtcatttccggctgctgtgtgcgagcagtggacacgtgagagagggggagagagattgagtgagagagagaggagaatgagagagaccacgagctcggacttaactaccctcgctagataatctttctgaaagccaacgagctagggggcatggtatgaagacccagtttgttgaagaattgcacatagatgttgaatgaatgaatgtaacccaaacaggaccctaaccgcctcccccccaaacccccccatccctcttcagtgaaaaatctttgttctttgtttgtttgtttctcttaatgcactggacagtgacagctacacacagtccccataaggatacccaagtgtggggtccaacattcttgaagttgtgttgaatcatatgctttttcacttttgatatataaacaagcaaaatattttttaaaaataataaaataagttaattaaaatataaaaaaaaaaaaaaaaaa

SEQ ID NO: 8 - protein, 400 aa, Mink
MQMSTVCLALGLALVFGEASASYLHETRAAELATDFGVKVFKQVAQASKDRNMVFSPYGLASVLAMLQLTTAGETRQQIQEAMRFQIDEKGMAPALRQLYKELMGPWNKDEISTADAIFVQRDLKLVHGFMPYFFRLFQTTVKQVDFSEVERARFIINDWVKRHTKGMIGDLLGRGTVDQLTRLMLVNALYFNGQWKTPFPKSGTHHRLFHKSDGSTVSVPMMAQTNKFNYTEFSTPEGRYYDILELPYHGDTLSMFIAAPYEKDVPLSALTNILDAQLISQWKGNMTRRLRLLVLPKFSLESEVNLRGPLENLGMTDMFRPNQADFSSLSDQEALYVSQALQKVKIEVNESGTVASSSTAIIVSARMAPEEIIMDRPFLFVVRHNPTGTVLFMGQVMEP

SEQ ID NO: 9 - DNA, 2999 bp, Mouse
  CDS (132)..(1340)
tcaggctgcagcagagccccgagagctttgtgaaggaggaccgccgcacacccgcctccggcacacacagccaaccacagctgagcgacagccaacaagagccaatcacaaggcacctttgaatactcaggatgcagatgtcttcagcccttgcttgcctcatcctgggcctggttctggtctctgggaaagggttcactttacccctccgagaatcccacacagcccatcaggccaccgacttcggagtaaaagtgtttcagcaggtggtccaggcctccaaagaccggaatgtggtcttctctccctatggcgtgtcctcggtgctggctatgctgcagatgaccacagcggggaaaacccggcggcagatccaagatgctatgggattcaaagtcaatgagaagggcacagctcatgccctccgccagctctccaaggagctcatggggccgtggaacaagaatgagatcagtactgcggatgccatctttgtccagcgggacctagagctggtccagggcttcatgccccacttcttcaagctcttccagactatggtgaaacaggtggacttctcagaggtggaaagagccagatttatcatcaatgactgggtggaaaggcataccaaaggtatgatcaatgacttactggccaagggggctgtagacgagctgacacgcctggtgctggtgaatgccctctacttcagtggccaatggaagacccctttcttagaggccagcacccaccagcgcctcttccacaagtctgatggcagcaccgtctctgtgcccatgatggctcagagcaacaagttcaactacactgagttcaccacccccgatgggctcgagtatgacgtcgtggaactgccctaccagcgggacaccctcagcatgttcatcgctgcaccctttgagaaagatgtgcacctctccgccctcaccaacatcttggatgctgaactcatcagacaatggaagggcaacatgaccaggctgccccgcctcctcatcctgcctaagttctctctggagactgaagtggacctcagagggcccctggagaagttgggcatgcctgacatgtttagtgcaaccctggccgacttcacaagtctttccgaccaagagcagctctctgtagcacaggcactgcaaaaggtcaggatcgaggtaaacgagagcggcacagtggcgtcttcctccacagcctttgtcatctcagcccgcatggcccccacggagatggttatagaccgatcctttctctttgtggttcggcacaacccgacagagacaatcctcttcatggggcaagtgatggagccttgacagtgggaagagacgccttcatttggacgaaactggagatgttataagcagaaactctgaagaaaaggttatttaaaggactctatggggagaaagagaaggcaactcctccttaccccccacactggtaatctttccaaccagcatcccagacctcggactcttgaagggaaaagagtctaactccctcctccctagggattcctaccccacaaaggtctcatggaccatagaactcacagtacctggatctgcccagcatgccctttggacccagttcccaccgaggccccagcagagtggagggcacaacactttcattcagcaaaatcgtttgtgttccagtcacactgtgggcacctcttgcatcgcctgccattgctgtggagggtgccatgggccaaaggaaaaagcactgtcctatctcaaggtccactgtggaaatgtccaccttgcccacctccaaggggcaacggatagacagatcaaatggtggcccaatagcgagccttctccctgctccctcccttgacacagcttgcttatgttatttcagagtgtaggtgacttgtttacacagcttttttcgacccacaaacttttttcatttggaaagggtgtaagaaaagtcggacgtgtgtgtgcctggctcttcgtccccagtctcccagtgggggggccctggggagattccaggggtgtgattgaatatttatctcttgctcttgtatgtttgttggggagaagaagcacttttaaggaaaatgcttcttatttaaaccgtggcatacggcatcccatttggggtctgcatccctgtatgtcaggggtgcatcactccacaaacctgcccctctgggtagcctcgtgatggggctcacactgccgcctagtggcagccgaacacacccttacccggtccctccctccctccccccccccccccccccccgtggctctttttccttagggaccttgccaaggtgatgcttggcaacccacgttaaaggaaggggggaaaaaagattagatggaagagagagagatttgagagagggcaaagtggtttcaaatttttccaaggcatccagaagcagagagggaaaaggggctgtgtgacctaacaggacagaactttctccaattactgggtgagtcagagctgcactggtgactcacttcaatgtgtcatttccggctgctgtatgtgagcagtggacacgtgggggggcgggggggggatgaaagagacagcagctcctggtcaaccaccttagttagataatcttttttgaaagcttcctagctggaggtatgatcagaaaaccaatttactgaaaaactgcacaagaaggtaccgtgaatgaatttcctagcaggccactctgcatctgttatgtctccaccggaaaaaaaataatcatgttggtgtttttgcttttctctctctccctctttctctctgatttttttttcctctcttttcattatgcactggacagccacacaccgtgtaccatagggccccaaatgtggggtcacatggtcttgaattttgttggttacatatgcctttttgttgttgtttgtcttcacttttgatatataaacaggtaaatatgttttttaaaaaatactaaatatagagaatatgcaaac

SEQ ID NO: 10 - protein, 402 aa, Mouse
MQMSSALACLILGLVLVSGKGFTLPLRESHTAHQATDFGVKVFQQVVQASKDRNVVFSPYGVSSVLAMLQMTTAGKTRRQIQDAMGFKVNEKGTAHALRQLSKELMGPWNKNEISTADAIFVQRDLELVQGFMPHFFKLFQTMVKQVDFSEVERARFIINDWVERHTKGMINDLLAKGAVDELTRLVLVNALYFSGQWKTPFLEASTHQRLFHKSDGSTVSVPMMAQSNKFNYTEFTTPDGLEYDVVELPYQRDTLSMFIAAPFEKDVHLSALTNILDAELIRQWKGNMTRLPRLLILPKFSLETEVDLRGPLEKLGMPDMFSATLADFTSLSDQEQLSVAQALQKVRIEVNESGTVASSSTAFVISARMAPTEMVIDRSFLFVVRHNPTETILFMGQVMEP

SEQ ID NO: 11 - DNA, 2999 bp, Mouse; identical to SEQ ID NO: 9
  CDS (132)..(1340)

SEQ ID NO: 12 - protein, 402 aa, Mouse; identical to SEQ ID NO: 10

SEQ ID NO: 13 - DNA, 3053 bp, Rat
  CDS (119)..(1327)
cccccgagagctttgtgaaggaggaacgctgcacacccgcctcccgcagcacacagccaaccacagctgagcgacacgcaacaagagccaatcacaaggcacttccgaaagctccaggatgcagatgtcttcagccctcacttgcctcaccctgggcctggttctggtctttgggaaagggttcgcttcaccccttccagagtcccatacagcccagcaggccaccaacttcggagtaaaagtgtttcagcatgtggtccaggcctccaaagaccgaaatgtggtcttctctccctacggcgtgtcctcggtgctggctatgctgcagctgaccacagcagggaaaacccggcagcagatccaagatgctatgggattcaatatcagtgagaggggcacagctcctgccctccgaaagctctccaaggagctcatggggtcatggaacaagaatgagatcagtactgcggacgccatctttgtccagcgggacctagagctggtccagggcttcatgccccacttcttcaagctcttccggaccacggtgaagcaggtggacttctcagaggtggaaagagccagattcatcatcaacgactgggtggagaggcacaccaaaggtatgatcagtgacttactggccaagggggctgtaaatgagctgacacgcctggtgctggtgaacgccctctatttcaacggccaatggaagacccccttcttagaggccagcacccaccagcgcctgttccacaagtctgatggtagcaccatctccgtgcccatgatggctcagaacaacaagttcaactacactgagttcaccactccggatgggcacgagtacgacatcctggaactgccctaccacggcgaaaccctcagcatgttcattgcagcaccctttgaaaaagatgtgcccctctccgccatcaccaacattttggacgctgagctcatcagacaatggaagagcaacatgaccaggctgccccgcctcctcatcctgcctaagttctctctggagactgaagtggacctcagagggcccctggagaagctgggcatgactgacatcttcagctcaacccaggccgacttcacaagtctttccgaccaagagcagctctctgtagcacaagcactacaaaaggtcaagatcgaggtgaacgagagcggcacagtggcgtcttcctccacagccattctagtctcagcccgcatggcccccacggagatggttttagaccgatcctttctctttgtggttcggcacaatccaacagagacaatcctcttcatgggccagctgatggagccttgagagtgggatgagaagcctttcctttgggacaaaactggacgtgttataagcagagactctgaagaaaagaattgttttaaggactctttggggagaaagagaaggcctttctttcttaccccggcactggtaaatctttccaaccagcctcccagacctcagactctcgaagaggaaagagtctaactccctcactagggacctatcttactaaggtctcatccaaccatagaactcacagaatctggatctgcccagcattcagcctttggacccagttcccaccaaggccccagcagggccaacccactacgccttcactcagcaaagtcttttgtgttccagtcacactctgggtacctcttgtatcgtcctccattgctatgaaggatgacccaggccaaaggaagaagcactgtcctatctcaaggtccactgtggaaatgaacaccttgcccatccccaaggggcagcagatagacagatcgaatgatcgcccgatatcaagccttctcccagctcccgtcctgccctcccttccctgacagccgccttgtgttatttcagagtgtagatgacttgtttacagcttttttcgacccacaaacttttctcattttgaaagcgtgaaagaaaggtcagatgtgcacgtgccttgctctttatcctgggtctccctgtgaggggagaggggtcctggggagattccaggggtgtgattgaatatttatcttgtttatcttatacgtttgttggggagaagaagcactattaaggagaaagccttttatttaaaccatggcatatggtgtcccatttggggtctgtatccctgtatgtcagggaggcatcactccacaaacccgcccctcgggtggcccggcgtcggggctcacactgccgcctagtggcagccgaacacgcccttgccccatccctcccccgcatcctcccccgtggctcttttccttagggatcttgccaaggtgatgcttggcagcccacggtaaaggaagggggaaaaagattaggtgggagagagagagagagagagagagagagagagagagagagagagagagagagagagagagagagagagaaagagagagagatgtttgagagagggcaaagtggtttcaaatttttccaatacattcagaagccgagtgggaaagggggctgtgtgacctaacaggacagaactttctccaattactgggtgactcagctgcactggtgactcacttcaatgtgtcatttccggctgctgtaagtgagcagtggacacgtgggggggggggggtgaggatgaaagaaacagccagctcctggtcaaccaccttagttagataatcttttttgaaagcttcctagctgggggtatgatcagaaaaccaatttactgaaaaactgcacaggaaggtaacgtgaatctaatttcatagcgggccgctctgcatccgttacatctccactggaaaaaaataatcattttctttttgtgtgtgtgtgtgtgttttagcttttctccctctccctctttctctctcatttcattatgcactggataaccatacaccgtgtaccacaggggcccaaatgtggggtcacatggtcttgaattttgtggggtacatatgcctttgtttgtttgttttcacttttgatatataaacaggtaaatgtgtttttaaaaaataataaaaatagagaatatgcagac

SEQ ID NO: 14 - protein, 402 aa, Rat
MQMSSALTCLTLGLVLVFGKGFASPLPESHTAQQATNFGVKVFQHVVQASKDRNVVFSPYGVSSVLAMLQLTTAGKTRQQIQDAMGFNISERGTAPALRKLSKELMGSWNKNEISTADAIFVQRDLELVQGFMPHFFKLFRTTVKQVDFSEVERARFIINDWVERHTKGMISDLLAKGAVNELTRLVLVNALYFNGQWKTPFLEASTHQRLFHKSDGSTISVPMMAQNNKFNYTEFTTPDGHEYDILELPYHGETLSMFIAAPFEKDVPLSAITNILDAELIRQWKSNMTRLPRLLILPKFSLETEVDLRGPLEKLGMTDIFSSTQADFTSLSDQEQLSVAQALQKVKIEVNESGTVASSSTAILVSARMAPTEMVLDRSFLFVVRHNPTETILFMGQLMEP

SEQ ID NO: 15 - DNA, 16 bp, Mouse
ctagagtcggggcggc

SEQ ID NO: 16 - DNA, 28 bp, Mouse
cttatcgattttaccacatttgtagagg